(19) United States
(12) Patent Application Publication
(10) Pub. No.: US 2025/0295458 A1
(43) Pub. Date: Sep. 25, 2025

(54) Surgical Tracker System Utilizing A Digital Display Screen

(71) Applicant: Stryker Corporation, Portage, MI (US)

(72) Inventors: Matthew Ervin Harrow, Mooresville, NC (US); Florian Herrmann, Schwanau (DE); Mayank Kumar, San Jose, CA (US)

(73) Assignee: Stryker Corporation, Portage, MI (US)

(21) Appl. No.: 19/082,541

(22) Filed: Mar. 18, 2025

Related U.S. Application Data
(60) Provisional application No. 63/567,062, filed on Mar. 19, 2024.

(51) Int. Cl. A61B 34/30; A61B 34/20

(52) U.S. Cl. CPC A61B 34/30; A61B 34/20; A61B 2034/2055; A61B 2034/305

(57) ABSTRACT

Systems, methods, non-transitory computer-readable mediums, and techniques involve a tracker system including a tracker device and a controller. The tracker device is attached or attachable to a surgical object. The tracker device includes a digital display screen. The controller is coupled to the tracker device and is configured to present, on the digital display screen, a computer-generated content, such as surgical information and/or a trackable graphic that is detectable by a tracking system to facilitate tracking of a pose of the surgical object.

### CROSS-REFERENCE TO RELATED APPLICATIONS

The subject application claims priority to and all the benefits of U.S. provisional patent application No. 63/567,062, filed Mar. 19, 2024, the entire contents of which are hereby incorporated by reference.

### BACKGROUND

Surgical navigation involves the tracking of objects in the operating room, such as the patient, surgical tools, etc. Typically, a tracker is attached to each object being tracked. The trackers are usually optically based and include either passive (retro-reflective) markers or active (e.g., LED) markers, which reflect/transmit infrared signals to sensors of an optical localizer of a navigation system. To distinguish between objects, each tracker typically includes a unique arrangement of markers. In turn, each tracker requires a mechanical support structure uniquely sized for the specific marker arrangement. Although active markers (LEDs) can change their transmission sequence or power, the markers must remain mechanically fixed to the tracker support structure.

More recent developments in tracking technology involves placing a QR code marker on surface of the tracker that is coupled to the object. A camera system, such as a camera of an optical localizer of a navigation system or a camera of a head-mounted device, detects the QR code marker to track the object. The QR code marker is a passive target that is merely printed on, etched, or adhered to the surface of the tracker, like a sticker.

The conventional trackers and markers (optical or QR code markers) described above have several shortcomings. Firstly, a unique tracker must be created for each different object that requires tracking. Many trackers, particularly those with optical tracking markers, have many parts that must be assembled and separately sterilized. This adds complexity and cost to the surgical system.

Also, such markers are not intelligently controllable to adapt to the dynamic conditions in the operating room. The tracker configurations are pre-set and cannot adapt or change. For example, conventional markers are fixed to their support structures. As such, the poses (position or orientation), shape, or arrangement of these markers cannot be actively controlled or changed relative to their support structures. If the tracker is placed in a sub-optimal manner, the respective markers will also be sub-optimally placed. If a tracker is rotated relative to the camera, the markers may lose visibility to the camera. As such, conventional trackers are quite susceptible to tracking inaccuracies and losing line-of-sight to the camera seeking to track the object.

Furthermore, conventional trackers seriously lack the ability to provide functionality beyond merely tracking the object. A user has no ability to communicate or interact with such trackers. Conventional trackers are neither capable of communicating surgically meaningful information (such as text, graphics, or video) nor intelligently adjusting to conditions that may affect tracking.

### SUMMARY

This Summary introduces a selection of concepts in a simplified form that are further described below in the Detailed Description below. This Summary is not intended to limit the scope of the claimed subject matter nor identify key features or essential features of the claimed subject matter.

According to a first aspect, a tracker system is provided comprising: a tracker device being attached or attachable to a surgical object, wherein the tracker device comprises a digital display screen; and a controller coupled to the tracker device, wherein the controller is configured to present, on the digital display screen, a computer-generated trackable graphic that is configured to be detectable by a tracking system to facilitate tracking of a pose of the surgical object.

According to a second aspect, a surgical tracker is provided comprising a housing that is attached or attachable to a surgical object, wherein the housing supports a digital display screen that is configured to generate a computer-generated trackable graphic to facilitate tracking of a pose of the surgical object.

According to a third aspect, a surgical tracker is provided comprising a housing that is attached or attachable to a surgical object, wherein the housing supports a controller and a digital display screen, wherein the controller controls the digital display screen to generate a computer-generated trackable graphic to facilitate tracking of a pose of the surgical object.

According to a fourth aspect, a surgical system is provided comprising: a tracking system, and a tracker device being attached or attachable to a surgical object, wherein the tracker device comprises a digital display screen; and a controller coupled to the tracker device, wherein the controller is configured to present, on the digital display screen, a computer-generated trackable graphic, and wherein the tracking system is configured to detect the computer-generated trackable graphic to facilitate tracking of a pose of the surgical object.

According to a fifth aspect, a tracking control system is provided comprising: a controller configured to communicate to a tracker device that includes a digital display screen to cause the tracker device to display a computer-generated trackable graphic on the digital display screen.

According to a sixth aspect, a surgical tracker system is provided comprising: a tracker device being removably attachable to a surgical object, wherein the tracker device comprises a digital display screen that presents computer-generated information that is configured to facilitate tracking of a pose of the surgical object.

According to a seventh aspect, a surgical tracker system is provided comprising: a tracker device being attached or attachable to a surgical object, and a controller coupled to the tracker device, wherein the tracker device is trackable to facilitate tracking of a pose of the surgical object, and wherein the tracker device comprises a digital display screen and the controller is configured present computer-generated surgical information on the digital display screen.

According to an eighth aspect, a surgical tracker system is provided comprising: a tracker device being attached or attachable to a surgical object, and a controller coupled to the tracker device, wherein the tracker device is trackable to facilitate tracking of a pose of the surgical object, and wherein the tracker device comprises a digital display screen and the controller is configured present a video stream related to a surgical procedure on the digital display screen.

According to a ninth aspect, a surgical tracker system is provided comprising: a tracker device being attached or attachable to a surgical object, wherein the tracker device comprises a plurality of infrared markers and a digital display screen configured to present a computer-generated trackable graphic, wherein the infrared markers and the computer-generated trackable graphic are detectable by one or more tracking systems to facilitate tracking of a pose of the surgical object.

According to a tenth aspect, a surgical tracker system is provided comprising: a tracker device being attached or attachable to a surgical object to facilitate tracking of a pose of the surgical object, wherein the tracker device comprises a digital display screen; and a controller coupled to the tracker device, wherein the controller is configured to: detect a condition or event; and generate computer-generated content related to the condition or event for presentation on the digital display screen.

According to an eleventh aspect, a surgical tracker system is provided comprising: a first tracker device being attached or attachable to a first surgical object to facilitate tracking of a pose of the first surgical object, wherein the first tracker device comprises at least one first digital display screen; a second tracker device being attached or attachable to a second surgical object, wherein the second tracker device comprises at least one second digital display screen; and one or more controller coupled to the first and second tracker devices, wherein the one or more controllers are configured to coordinate presentation of computer-generated content on the at least one first and second digital display screens in response to detection of a condition or event.

According to a twelfth aspect, a surgical tracker system is provided comprising: a first tracker device being attached or attachable to a first surgical object to facilitate tracking of a pose of the first surgical object, wherein the first tracker device comprises a camera; a second tracker device being attached or attachable to a second surgical object, wherein the second tracker device comprises a digital display screen to present a computer-generated trackable graphic to facilitate tracking of a pose of the second surgical object; one or more controller coupled to the first and second tracker devices, wherein the one or more controllers are configured to utilize the camera of the first tracker device to detect the computer-generated trackable graphic presented by the second tracker device.

Also provided are: a method of operating any one or more of: the tracker system of the first aspect, the surgical tracker of the second aspect, the surgical tracker of the third aspect, the surgical system of the fourth aspect, the tracking control system of the fifth aspect, or the tracker system of any of the sixth-twelfth aspects.

Also provided are: a non-transitory computer readable medium comprising instructions, which when executed by one or more processors, implement operation of any one or more of: the tracker system of the first aspect, the surgical tracker of the second aspect, the surgical tracker of the third aspect, the surgical system of the fourth aspect, the tracking control system of the fifth aspect, or the tracker system of any of the sixth-twelfth aspects.

Any of the above aspects may be combined, in whole or in part.

Any of the above aspects may be combined with any of the following implementations. Any of the following implementations may be utilized in part, or in whole, with any of the above aspects. The implementations are:

The pose of the computer-generated trackable graphic can be dynamically changed on the digital display screen. The pose of the computer-generated trackable graphic on the digital display screen can be changed depending on a relative spatial relationship between the tracker device and the tracking system. The pose of the computer-generated trackable graphic on the digital display screen can be changed to react to an absence or presence of line-of-sight between the tracker device and the tracking system. The location of the computer-generated trackable graphic on the digital display screen can be changed to react to a movement of the tracker device. Measurements from a time-of-flight sensor can be utilized to determine the relative spatial relationship between the tracker device and the tracking system. The computer-generated trackable graphic can be presented in a manner configured to enable the tracking system to determine the pose of the surgical object in at least five-degrees of freedom. The computer-generated trackable graphic can be generated based on surgical information. The surgical information can include but is not limited to: information about the surgical object, an identity of the surgical object, information about a surgical procedure or step of the surgical procedure, surgical plan information, surgeon preferences, a tracking status of the tracker device, an operation status of the tracking system, and a location or the tracking system. The surgical information can be utilized to query a database of predetermined computer-generated trackable graphics and retrieve a predetermined computer-generated trackable graphic from the database. The predetermined computer-generated trackable graphic can be presented on the digital display screen. The computer-generated trackable graphic can be encoded with time stamps to facilitate synchronization with the tracking system. A configuration or geometry of the computer-generated trackable graphic is configured to change, e.g., during use of the tracker device. A successful registration of the tracker device to the surgical object can be detected or inputted and the computer-generated trackable graphic can be presented on the digital display screen in response to successful registration. An identify or type of the surgical object can be detected or received, and the computer-generated trackable graphic can be presented on the digital display screen the computer-generated trackable graphic can be presented on the digital display screen in response to successful registration. The computer-generated trackable graphic can be a geometric array of at least three digital fiducials. The computer-generated trackable graphic can be a QR code or dynamic QR code. The tracker device can also support active or passive infrared markers to provide supplemental or redundant tracking.

Human-readable information can also be presented on the digital display screen. The human-readable information can include but is not limited to any one or more of: surgical information, operating instructions, information about the surgical object, an identity of the surgical object, a tracking status of the tracker device, an operation status of the tracking system, information about a surgical procedure or step of the surgical procedure, surgical plan information, and a warning, error, or alert related to the surgical procedure. The tracker device can implement a graphical user interface. The graphical user interface can enable a user to provide input, for example, to modify settings or operation of the tracker device or surgical object. Graphical information can be presented on the digital display screen. The graphical information can include but is not limited to any one or more of: information of or about the surgical object, information of or about any surgical object, medical imaging data, video data from a camera, elements or icons of a graphical user interface, a video stream provided from a software application of a device in the operating room, a warning, error, or alert information.

An attachment can be provided to enable the tracker device to be releasably coupled to the surgical object. The tracker device can be a portable electronic device that has capabilities beyond being used as a tracker device. The attachment can be a holder for the portable electronic device. The attachment can be a kinematic attachment. The tracker device can be clamped, pinned, mounted, fastened, or secured to the surgical object in any manner.

The tracker device is attached or attachable to any of the following surgical objects, including but not limited to: a patient anatomy, a surgical instrument, a surgical robot, (including any portion of the robot, such as the base, one or more links, the end effector body, the cart, or the like), a second tracker device, a navigation system, a head-mounted device, and an imaging device.

The digital display screen can be curved, and the computer-generated trackable graphic can be presented in a curved manner on the digital display screen. The computer-generated trackable graphic can be configured to move about the curved digital display screen. The curved digital display screen can be spherical, semi-spherical, cylindrical, or semi-cylindrical. The digital display screen can be an LED or OLED display screen. The digital display screen can have a resolution of at least 200 pixels by 200 pixels. The digital display screen can be a touch-screen controllable display. Multiple digital display screens can be arranged to face different directions from one another.

The computer-generated trackable graphic can be remotely detectable by any camera source, such as but not limited to a camera of a surgical head-mounted device or a camera of a surgical navigation system. The controller can be remote from the tracker device. The controller can be integrated with the tracker device.

The tracker device can include an inertial sensor from which measurements can be utilized to perform any one or more of the following: change an orientation of the computer-generated trackable graphic on the digital display screen; communicate the measurements to the tracking system to supplement tracking of the tracker device; detect that the tracker device is being moved by a user; and/or detect an undesired motion of the tracker device. The tracker device can include a camera configured to capture image or video data, and the image or video data can be utilized to perform one or more of the following: detect an event and modify the computer-generated trackable graphic in response; detect presence or absence of the surgical object; detect presence or absence of the tracking system; present the image or video data on the digital display screen; and/or detect a face of a user to authenticate use of the tracker device. The tracker device can include an infrared or radio frequency transceiver, and wherein the controller is configured to utilize the transceiver to communicate to a transceiver of the tracking system. The tracker device can include a proximity sensor that can be utilized to perform one or more of the following: detect absence of environmental activity and in response place the tracker device or digital display screen in a sleep mode to conserve energy; and/or detect presence of environmental activity to ensure the tracker device or digital display screen is active.

The tracker device is configured to interface with a surgical drape. The surgical drape can include a transparent window configured to cover the digital display screen. The transparent window can be coupled or configured to couple to a surgical drape. The tracker device can include a housing that supports a drape attachment mechanism. The drape attachment mechanism can enable attachment of the surgical drape to the tracker device. The tracker device can include a housing that defines a channel that surrounds the digital display screen. The channel can receive an elastic member to secure the surgical drape over the digital display screen. The controller can comprise a PCB that is Parylene coated and configured to withstand sterilization. The digital display screen can be removable with the controller from the housing of the tracker and the housing of the tracker can be sterilizable. A sterile cover, sticker, or sheet can be placed over the digital display screen. Any of the above implementations can be performed automatically. Any of the above implementations may be utilized in part, or in whole.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a surgical system implementing various tracking systems for detecting various tracker devices with digital display screens, according to one implementation.

FIG. 2 is a schematic view of an example control system can that be used with the surgical system.

FIG. 3 is a perspective view of a hand-held surgical tool comprises the tracker device with the digital display screen integrated into the surgical tool, according to one implementation.

FIG. 4 is a perspective view of the tracker device with the digital display screen removably attached to a patient anatomy, according to one implementation.

FIGS. 5A-5F illustrate several implementations of tracker devices with the digital display screens.

FIG. 6 is an example method of configuring operation of the tracker device based on various information sources and types, according to one implementation.

FIG. 7 is a perspective view illustrating an example operation of the tracker device attached to the patient, wherein the digital display screen is presented in an enlarged view, according to one implementation.

FIG. 8 is a perspective view illustrating another example operation of the tracker device attached to a surgical tool, wherein the digital display screen is presented in an enlarged view, according to one implementation.

FIGS. 9A and 9B illustrate an example by which a head-mounted device HMD tracking system observes the digital display screen of the tracker device from different perspectives, according to one implementation.

FIG. 10 is a perspective view illustrating an example by which a camera of navigation tracking system and an HMD tracking system simultaneously observe different digital display screens of one tracker device attached to the patient, according to one implementation.

FIG. 11 is a perspective view illustrating another example by which a camera of navigation tracking system simultaneously observes a digital display screen of a first tracker device attached to the patient and a digital display screen of a second tracker device attached to a surgical tool, wherein the digital display screen of the first tracker device is presented in an enlarged view, according to one implementation.

FIG. 12 is an example first-person view illustrating a user's hand interacting with a GUI implemented on the digital display screen of the tracker device, according to one implementation.

### DETAILED DESCRIPTION

### I. Example System Overview

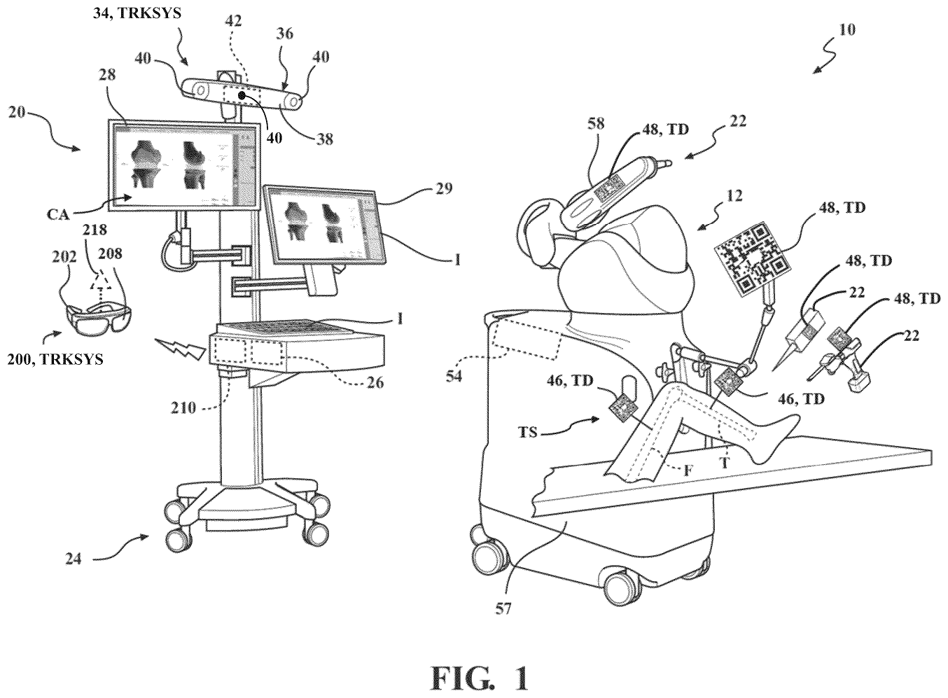

Referring to FIG. 1, a system 10 is provided. The system can be a surgical system 10 adapted for treating a target site TS of a patient. The surgical system 10 is shown in a surgical setting such as an operating room of a medical facility. The surgical system 10 may be used to perform any intraoperative surgical procedure on a patient. Example surgical procedures include, but are not limited to: partial knee arthroplasty, total knee arthroplasty, total hip arthroplasty, shoulder arthroplasty, spinal procedures, ankle procedures, endoscopic procedures, cranial procedures, lesion removal procedures, arthroscopic procedures, arthroscopic resection procedures, soft tissue or ligament repair procedures, neurological procedures, ENT procedures, minimally invasive MIS procedures, or the like. In the example shown in FIG. 1, the patient is undergoing a knee procedure. In addition, the following implementations describe the use of the surgical system 10 in performing a procedure in which material is removed from a femur F and/or a tibia T of a patient. However, it should be recognized that the surgical system 10 may be used to perform any suitable procedure in which material is removed from any suitable portion of a patient's anatomy, material is added to any suitable portion of the patient's anatomy (e.g., an implant, graft, etc.), and/or in which any other control of and/or visualization of a surgical tool is desired.

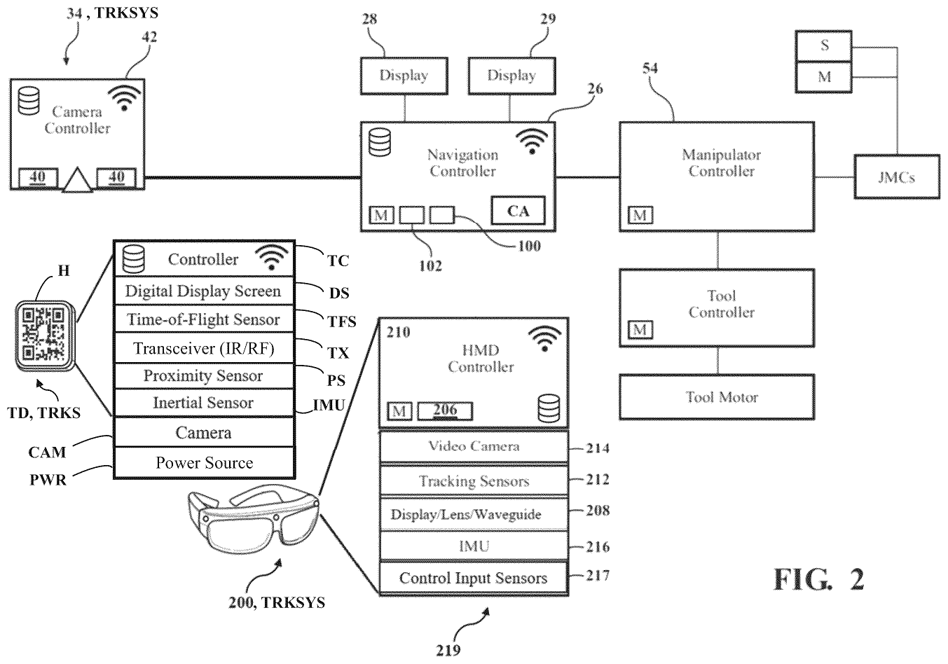

In the implementation shown, the surgical system 10 can include a manipulator 12 (e.g., surgical robot) and a navigation system 20. The navigation system 20 is set up to track movement of various objects in the operating room. Such objects include, for example, a surgical tool 22, a femur F of a patient, and a tibia T of the patient. The navigation system 20 can track these objects for purposes such as displaying their relative positions and orientations to the surgeon on a clinical application (CA) and, in some cases, for purposes of controlling or constraining movement of the surgical tool 22 relative to virtual cutting boundaries (VB) associated with the femur F and tibia T. An example control scheme for the surgical system 10 is shown in FIG. 2.

In the implementation shown, the surgical tool 22 is attached to the manipulator 12. Such an arrangement is shown in U.S. Pat. No. 9,119,655, entitled, “Surgical Manipulator Capable of Controlling a Surgical Instrument in Multiple Modes,” the disclosure of which is hereby incorporated by reference. In one example, the manipulator 12 has a base 57, a plurality of links 58 extending from the base 57, and a plurality of joints (not numbered) for moving the surgical tool 22 with respect to the base 57. The links 58 and joints form a robotic arm. Some or all of the joints may be passive joints or active joints. The manipulator 12 may have a serial arm or parallel arm configuration. The manipulator 12 can be floor mounted, ceiling mounted, gantry mounted, table mounted, or patient mounted. More than one manipulator 12 can be utilized.

While the surgical system 10 is illustrated in FIGS. 1-3 as including the surgical tool 22 attached to the manipulator 12, it should be recognized that the surgical system 10 may additionally or alternatively include one or more manually operated or hand-held surgical tools 22. For example, the surgical tool 22 may include a hand-held motorized saw, drill, bur, probe, or other suitable tool that may be held and manually operated by a surgeon. Any implementations described with reference to the use of the manipulator 12 may also apply to the use of a hand-held tool 22 with appropriate modifications. The surgical tool 22 may have working end or an energy applicator, such as a rotating bur, saw, router, reamer, impactor, electrical ablation device, cut guide, tool holder, probe, or the like. In other examples, the surgical tool 22 may be a camera tool, such as an endoscope, a laparoscope, an arthroscope, or a microscope. Any of the surgical tools 22 could be supported and moved by the manipulator 12.

The navigation system 20 can include one or more computer cart assemblies 24 that houses one or more navigation controllers 26. A navigation interface is in operative communication with the navigation controller 26. The navigation interface includes one or more displays 28, 29 adjustably mounted to the computer cart assembly 24 or mounted to separate carts as shown. Input devices I such as a keyboard and mouse can be used to input information into the navigation controller 26 or otherwise select/control certain aspects of the navigation controller 26. Other input devices I are contemplated including a touch screen, a microphone for voice-activation input, an optical sensor for gesture input, and the like.

The clinical application CA can be displayed on one or more displays 28, 29 of the navigation system 20. The clinical application CA assists a surgeon or staff in performing the surgical procedure. The clinical application CA can have a plurality of different screens related to the surgical procedure. Such screens can include a pre-operative planning screen, an operating room setup screen, an anatomical registration screen, an intra-operative planning screen, an anatomical preparation screen, or a post-operative evaluation screen, and the like. The clinical application CA can present a navigation guidance region that displays one or more of the surgical objects tracked by a localizer 34 of the navigation system 20.

The localizer 34 communicates with the navigation controller 26. In the implementation shown, the localizer 34 is an optical localizer and includes a camera unit 36. The camera unit 36 has a housing 38 comprising an outer casing that houses one or more optical sensors 40. The optical sensors 40 can detect light signals, such as infrared (IR) signals and/or visible light signals. Camera unit 36 can be mounted on an adjustable arm to position the optical sensors 40 with a field-of-view of the below discussed trackers that, ideally, is free from obstructions. The camera unit 36 includes a camera controller 42 in communication with the optical sensors 40 to receive signals from the optical sensors 40. The camera controller 42 communicates with the navigation controller 26 through either a wired or wireless connection. In other implementations, the optical sensors 40 communicate directly with the navigation controller 26. Position and orientation signals and/or data are transmitted to the navigation controller 26 for purposes of tracking objects. The computer cart assembly 24, display 28, and camera unit 36 may be like those described in U.S. Pat. No. 7,725,162 to Malackowski, et al. issued on May 25, 2010, entitled “Surgery System,” the disclosure of which is hereby incorporated by reference. The navigation controller 26 can be a personal computer or laptop computer. Navigation controller 26 includes the displays 28, 29, central processing unit (CPU) and/or other processors, memory (not shown), and storage (not shown). The navigation controller 26 is loaded with software that converts the signals received from the camera unit 36 into data representative of the position and orientation of the objects being tracked. The navigation controller 26 includes a navigation processor. It should be understood that the navigation processor could include one or more processors to control operation of the navigation controller 26. The processors can be any type of microprocessor or multi-processor system. The term processor is not intended to limit the scope of any implementation to a single processor.

Navigation system 20 is operable with a plurality of tracking devices 46, 48, also referred to herein as trackers. In the illustrated implementation, one tracker 46 can be firmly affixed to the femur F of the patient and another tracker 46 can be firmly affixed to the tibia T of the patient. Trackers 46 are firmly affixed to sections of bone in an implementation. For example, trackers 46 may be attached to the femur F and tibia T in the manner shown in U.S. Pat. No. 7,725,162 to Malackowski, et al. issued on May 25, 2010, entitled “Surgery System,” the disclosure of which is hereby incorporated by reference. Trackers 46, 48 may also be mounted like those shown in U.S. patent application Ser. No. 14/156,856, filed on Jan. 16, 2014, entitled, “Navigation Systems and Methods for Indicating and Reducing Line-of-Sight Errors,” hereby incorporated by reference herein. The trackers 46, 48 may be mounted to other tissue types or parts of the anatomy. One or more tool trackers 48 can be coupled to the manipulator 12, the end effector of the manipulator 12, or to the base of the manipulator 12. Tool trackers 48 can also be attached to any of the hand-held tools 22 at any suitable location. Any of these objects can be referred to as surgical tools 22. The tool tracker 48 can be integrated into the surgical tool 22 during manufacture or may be separately mounted to the surgical tool 22 in preparation for surgical procedures.

In one implementation, optical sensors 40 of the localizer 34 receive light signals from the trackers 46, 48. As will be described in detail below, any one or more of the trackers 46, 48 can be implemented with a digital display screen DS that can present a virtual object that can be detected by the localizer 34. Some of the trackers 46, 48 may include passive markers. For example, the tracker can have at least three passive tracking elements or markers (e.g., reflectors) for transmitting light signals (e.g., reflecting light emitted from the camera unit 36) to the optical sensors 40. In other implementations, some, or all of the trackers 46, 48 may include active tracking markers. The active markers can be, for example, light emitting diodes transmitting light, such as infrared light. Active and passive arrangements are possible. The camera unit 36 receives optical signals from the trackers 46, 48 and outputs to the navigation controller 26 signals relating to the position of the tracking markers of the trackers 46, 48 relative to the localizer 34. Based on the received optical signals, navigation controller 26 generates data indicating the relative positions and orientations of the trackers 46, 48 relative to the localizer 34. These relative positions can be displayed on the clinical application CA as graphical representations for surgical guidance.

Furthermore, in some examples, the navigation system 20 can additionally or alternatively implement the localizer 34 as a vision tracking system. The vision-based localizer 34 includes a vision or video camera coupled to the navigation controller 26. The vision camera can be the one or more of the optical sensors 40. The vision camera facilitates acquisition of 2D and/or 3D machine-vision images or view of structural features that define trackable features such that tracked states of the objects are communicated to (or interpreted by) the navigation controller 26 based on the machine-vision images or view. The machine vision system can be integrated into the camera unit 36, optionally in combination with infrared sensors. The machine vision system can create depth maps and can detect objects with or without trackers. The machine vision system can detect patterns, shapes, colors, computer-codes, tracking geometries, or the like.

Additionally, or alternatively, the navigation system 20 and/or the localizer 34 can employ radio frequency (RF) based tracking. For example, the navigation system 20 may comprise an RF transceiver coupled to the navigation controller 26. Here, the trackers 46, 48 may comprise RF emitters or transponders, which may be passive or may be actively energized. The RF transceiver transmits an RF tracking signal, and the RF emitters respond with RF signals such that tracked states are communicated to (or interpreted by) the navigation controller 26. The RF signals may be of any suitable frequency. The RF transceiver may be positioned at any suitable location to track the objects using RF signals effectively. Furthermore, examples of RF-based navigation systems may have structural configurations that are different than the navigation system 20 illustrated throughout the drawings.

Additionally, or alternatively, the navigation system 20 and/or localizer 34 can employs aspects of electromagnetic (EM) tracking. For example, the navigation system 20 may comprise an EM transceiver coupled to the navigation controller 26. Here, the trackers 46, 48 may comprise EM components attached thereto (e.g., various types of magnetic trackers, electromagnetic trackers, inductive trackers, and the like), which may be passive or may be actively energized. The EM transceiver generates an EM field, and the EM components respond with EM signals such that tracked states are communicated to (or interpreted by) the navigation controller 26. The navigation controller 26 may analyze the received EM signals to associate relative states thereto. Here too, examples of EM-based navigation systems may have structural configurations that are different than the navigation system 20 illustrated throughout the drawings.

In other examples, the navigation system 20 and/or the localizer 34 could be based on one or more other types of tracking systems. For example, an ultrasound-based tracking system coupled to the navigation controller 26 could be provided to facilitate acquiring ultrasound images of markers that define trackable features on the tracked objects such that tracked states are communicated to (or interpreted by) the navigation controller 26 based on the ultrasound images. By way of further example, a fluoroscopy-based imaging system (e.g., a C-arm) coupled to the navigation controller 26 could be provided to facilitate acquiring X-ray images of radio-opaque markers that define trackable features such that tracked states are communicated to (or interpreted by) the navigation controller 26 based on the X-ray images. The shape of the trackers 46, 48 can also be of a geometry that can be identified in X-ray imaging to assist in registration.

Several types of tracking and/or imaging systems could define the localizer 34 and/or form a part of the navigation system 20 without departing from the scope of the present disclosure. Furthermore, the navigation system 20 and/or localizer 34 may have other suitable components or structure not specifically recited herein, and the various techniques, methods, and/or components described herein with respect to the optically-based navigation system 20 shown throughout the drawings may be implemented or provided for any of the other examples of the navigation system 20 described herein. For example, the navigation system 20 may utilize solely inertial tracking and/or combinations of different tracking techniques, sensors, and the like. Any of the described tracking methods can be included in the trackers 46, 48. Other configurations are contemplated.

Based on the position and orientation of the trackers 46, 48 and previously loaded data, navigation controller 26 can determine the position and/or the orientation of the surgical tool 22 relative to the tissue against which the working end is to be applied. In some implementations, the navigation controller 26 forwards these data to a manipulator controller 54. The manipulator controller 54 can then use the data to control the manipulator 12. This control can be like that described in U.S. Pat. No. 9,119,655, entitled, “Surgical Manipulator Capable of Controlling a Surgical Instrument in Multiple Modes,” or like that described in U.S. Pat. No. 8,010,180, entitled, “Haptic Guidance System and Method”, the disclosures of which are hereby incorporated by reference.

In one implementation, the manipulator 12 is controlled to stay within a preoperatively defined virtual boundary VB that can be determined by a surgical plan. The virtual boundary VB may be a virtual cutting boundary which defines the material of the anatomy (e.g., the femur F and tibia T) to be removed by the surgical tool 22. For example, each of the femur F and tibia T may have a target volume of material that is to be removed by the working end of the surgical tool 22. The target volumes are defined by one or more virtual cutting boundaries. The virtual cutting boundaries define the surfaces of the bone that should remain after the procedure. The navigation system 20 tracks and controls the surgical tool 22 to ensure that the working end, e.g., the surgical bur, removes the target volume of material and does not extend beyond the virtual cutting boundary, as disclosed in U.S. Pat. No. 9,119,655, entitled, “Surgical Manipulator Capable of Controlling a Surgical Instrument in Multiple Modes,” the disclosure of which is hereby incorporated by reference, or as disclosed in U.S. Pat. No. 8,010,180, entitled, “Haptic Guidance System and Method”, the disclosure of which is hereby incorporated by reference.

The virtual cutting boundary VB may be defined within a virtual model of the anatomy (e.g., the femur F and tibia T), or separately from the virtual model. The virtual cutting boundary may be represented as a mesh surface, constructive solid geometry (CSG), voxels, or using other boundary representation techniques. The surgical tool 22 may be used to cut away material from the femur F and tibia T to receive an implant. The surgical implants may include unicompartmental, bicompartmental, or total knee implants as shown in U.S. Pat. No. 9,381,085, entitled, “Prosthetic Implant and Method of Implantation,” the disclosure of which is hereby incorporated by reference. Other implants, such as hip implants, shoulder implants, spine implants, and the like are also contemplated. The focus of the description on knee implants is provided as one example. These concepts can be equally applied to other types of surgical procedures, including those performed without placing implants.

The navigation controller 26 also generates image signals that indicate the relative position of the working end to the tissue. These images can be presented on the displays 28, 29 by the clinical application CA to allow the surgeon and staff to view the relative position of the working end to the target site TS.

Prior to the start of the intraoperative procedure, preoperative images of the femur F and tibia T may be generated (or of other portions of the anatomy in other implementations). The preoperative images can be stored as two-dimensional or three-dimensional patient image data in a computer-readable storage device, such as memory (M) within the navigation system 20. The patient image data may be based on X-ray scans or computed tomography (CT) scans of the patient's anatomy. The patient image data may then be used to generate two-dimensional images or three-dimensional models of the patient's anatomy. The pre-operative data and models may be used for purposes of surgical planning purposes and intraoperative guidance. For example, the surgical plan (e.g., tool path TP or resection volume or boundaries VB), may be planned relative to the virtual model. The virtual model and surgical plan can then be registered to the anatomy using any appropriate registration technique, such as pointer registration, imageless registration, or the like.

FIG. 2 illustrates a schematic view of an example control system that be used with the surgical system 10. A localization engine 100 is a software module that can be considered part of the navigation system 20. Components of the localization engine 100 run on navigation controller 26. In some implementations, the localization engine 100 may run on the manipulator controller 54. Localization engine 100 receives as inputs the signals from the localizer 34 and, in some implementations, signals from the trackers 46, 48. Based on these signals, localization engine 100 can determine the pose of each tracker coordinate system in the localizer coordinate system. The localization engine 100 forwards the signals representative of the poses of trackers 46, 48 to a coordinate transformer 102. Coordinate transformer 102 is a navigation system software module that runs on navigation controller 26. Coordinate transformer 102 references the data that defines the relationship between the preoperative images of the patient and the anatomy trackers 46. Coordinate transformer 102 can also store the data indicating the pose of the working end of the surgical tool 22 relative to a tool tracker 48. During the procedure, the coordinate transformer 102 receives the data indicating the relative poses of the trackers 46, 48 to the localizer 34. Based on these data, the previously loaded data, and encoder data from the manipulator 12, the coordinate transformer 102 generates data indicating the position and orientation of the working end of the surgical tool 22 relative to the tissue (e.g., bone) against which the working end is applied. Image signals representative of these data are forwarded to displays 28, 29 enabling the surgeon and staff to view this information. In certain implementations, other signals representative of these data can be forwarded to the manipulator controller 54 to guide the manipulator 12 and corresponding movement of the surgical tool 22.

The manipulator 12 has the ability to operate in a manual mode or a semi-autonomous mode in which the surgical tool 22 is moved along a predefined tool path, as described in U.S. Pat. No. 9,119,655, entitled, “Surgical Manipulator Capable of Controlling a Surgical Instrument in Multiple Modes,” the disclosure of which is hereby incorporated by reference, or the manipulator 12 may be configured to move in the manner described in U.S. Pat. No. 8,010,180, entitled, “Haptic Guidance System and Method”, the disclosure of which is hereby incorporated by reference.

The manipulator controller 54 can use the position and orientation data of the surgical tool 22 and the patient's anatomy to control the manipulator 12 as described in U.S. Pat. No. 9,119,655, entitled, “Surgical Manipulator Capable of Controlling a Surgical Instrument in Multiple Modes,” the disclosure of which is hereby incorporated by reference, or to control the manipulator 12 as described in U.S. Pat. No. 8,010,180, entitled, “Haptic Guidance System and Method”, the disclosure of which is hereby incorporated by reference.

The manipulator controller 54 may have a central processing unit (CPU) and/or other manipulator processors, memory, and storage. The manipulator controller 54, also referred to as a manipulator computer, is loaded with software as described below. The manipulator processors could include one or more processors to control operation of the manipulator 12. The processors can be any type of microprocessor or multi-processor system. The term processor is not intended to limit any implementation to a single processor.

Position sensors S can be associated with the plurality of links 58 of the manipulator 12. In one implementation, the position sensors S are encoders. The position sensors S may be any suitable type of encoder, such as rotary encoders. Position sensors S can be associated with a joint actuator, such as a joint motor M. The position sensor S is a sensor that monitors the angular position of one of six motor driven links 58 of the manipulator 12 with which the position sensor S is associated. Multiple position sensors S may be associated with each joint of the manipulator 12 in some implementations. The manipulator 12 can also include a force/torque sensor coupled between the distal end of the manipulator 12 and the end effector for detecting manual forces/torques exerted on the tool 22 by an operator. The input forces/torques can be used to command movement of the manipulator 12 and/or to detect collisions with the tool 22.

In some modes, the manipulator controller 54 determines the desired location to which the surgical tool 22 should be moved. Based on this determination, and information relating to the current location (e.g., pose) of the surgical tool 22, the manipulator controller 54 determines the extent to which each of the plurality of links 58 needs to be moved in order to reposition the surgical tool 22 from the current location to the desired location. The data regarding where the plurality of links 58 are to be positioned is forwarded to joint motor controllers JMCs that control the joints of the manipulator 12 to move the plurality of links 58 and thereby move the surgical tool 22 from the current location to the desired location. In other modes, the manipulator 12 is capable of being manipulated as described in U.S. Pat. No. 8,010,180, entitled, “Haptic Guidance System and Method”, the disclosure of which is hereby incorporated by reference, in which case the actuators are controlled by the manipulator controller 54 to provide gravity compensation to prevent the surgical tool 22 from lowering due to gravity and/or to activate in response to a user attempting to place the working end of the surgical tool 22 beyond a virtual boundary VB.

To determine the current location of the surgical tool 22, data from the position sensors S is used to determine measured joint angles. The measured joint angles of the joints are forwarded to a forward kinematics module, as known in the art. Based on the measured joint angles and preloaded data, the forward kinematics module determines the pose of the surgical tool 22 in a manipulator coordinate system. The preloaded data are data that define the geometry of the plurality of links 58 and joints. With this encoder-based data, the manipulator controller 54 and/or navigation controller 26 can transform coordinates from the localizer coordinate system into the manipulator coordinate system, vice versa, or can transform coordinates from one coordinate system into any other coordinate system described herein using transformation techniques. In many cases, the coordinates of interest associated with the surgical tool 22 (e.g., the tool center point or TCP), the virtual boundaries, and the tissue being treated, are transformed into a common coordinate system for purposes of relative tracking and display.

While the example surgical system 10 has been described with reference to the Figures, the surgical system 10 is not intended to be limited to what is specifically shown and described. For example, the surgical system 10 may not include the manipulator 12 or the navigation system 20 as specifically shown. Other systems are contemplated without departing from the scope of the disclosure.

### II. Head Mounted Device

Referring back to FIGS. 1 and 2, one or more head-mounted devices (HMDs) 200 may be incorporated into the surgical system 10. The HMD may be employed to enhance visualization before, during, and/or after surgery. The HMD 200 is an extended reality device, which can include aspects of augmented reality, mixed reality, virtual reality, and the like. The HMD 200 can be used to visualize the same objects previously described as being visualized on the displays 28, 29, and can also be used to visualize other objects, features, instructions, warnings, etc. The HMD 200 can be used to assist with visualization of the volume of material to be cut from the patient, to help visualize the size of implants and/or to place implants for the patient, to assist with registration and calibration of objects being tracked via the navigation system 20, to see instructions and/or warnings, among other uses, as described further below.

The HMD 200 has a display 208 onto which computer-generated content can be displayed onto a real-world view. In the implementation described herein, the HMD 200 provides on the HMD display 208 a computational holographic/superimposed/overlay of computer-generated content over the real-world view. In one example, the real-world view is acquired by a video camera 214 attached to the HMD. The video camera 214 produces a live video stream of the real-world and the computer-generated content may be combined into video stream of the real world. In such instances, the HMD display 208 may include one or more high-resolution displays positioned in front of the user's eyes. The HMD display 208 may be opaque in such scenarios.

In other implementations, the HMD 200 may implement natural see-through techniques whereby the HMD display 208 is implemented as a transparent lens/visor/waveguide provided between the user's eyes and the real-world. The real-world view is acquired naturally by the user's eyes and the computer-generated content is provided on the transparent lens/visor/waveguide. Such see-through techniques can include a diffractive waveguide, holographic waveguide, polarized waveguide, reflective waveguide, or switchable waveguide.

As shown in FIG. 1, the HMD 200 includes a support structure 202, which may be head-mountable in the form of an eyeglass or glasses, headwear or headset, or eyewear (such as a digital contact lens or lenses). The HMD 200 may include additional headbands or supports to hold the HMD 200 on the user's head. In other implementations, the HMD 200 may be integrated into a surgical helmet or other structure worn on the user's head, neck, and/or shoulders. Although not shown, it is contemplated that instead of the HMD 200, an extended reality display screen, such as a monitor, tablet, or hand-held display may be used, which can include similar hardware and capabilities as the described HMD 200.

As shown in FIG. 2, the HMD 200 can include an HMD controller 210. The HMD controller 210 can include a content generator 206 that generates the computer-generated content (also referred to as virtual images) and that transmits those images to the user through the HMD display 208. The HMD controller 210 controls the transmission of the computer-generated content to the HMD display 208. The HMD controller 210 may be a separate computer, located remotely from the support structure 202 of the HMD 200, or may be integrated into the support structure 202 of the HMD 200. The HMD controller 210 may be a laptop computer, desktop computer, microcontroller, or any suitable controller comprising memory (M), one or more processors (e.g., multi-core processors), input devices I, output devices (fixed display in addition to HMD 200), storage capability, etc.

The HMD 200 comprises a plurality of tracking sensors 212 that are in communication with the HMD controller 210. In some cases, the tracking sensors 212 are provided to establish a global coordinate system for the HMD 200, also referred to as an HMD coordinate system. The HMD coordinate system is established by these tracking sensors 212, which may comprise camera sensors or other sensor types, in some cases combined with IR depth sensors, to layout the space surrounding the HMD 200, such as using structure-from-motion techniques or the like. The HMD 200 can also comprise a photo/video camera 214 in communication with the HMD controller 210. The camera 214 may be used to obtain photographic images or video with the HMD 200, which can be useful in identifying objects or markers attached to objects, as will be described further below. The HMD 200 can comprise an inertial measurement unit IMU 216 in communication with the HMD controller 210. The IMU 216 may comprise one or more 3-D accelerometers, 3-D gyroscopes, and the like to assist with determining a position and/or orientation of the HMD 200 in the HMD coordinate system or to assist with tracking relative to other coordinate systems. The HMD 200 could have a speaker to generate a sound or vibrate to provide an indication to the HMD user of a warning or other information of relevance.

The HMD 200 may also comprise control input sensors 217. In one example, the control input sensors 217 are configured to recognize gesture or eye-based commands from the user. When detecting hand-gestures, the control input sensor 217 is able to sense the user's hands, fingers, or other objects for purposes of determining the user's gesture command and controlling the HMD 200, HMD controller 210, navigation controller 26, and/or manipulator controller 54 accordingly. Gesture commands can be used for any type of input used by the system 10. The gesture commands may be detected below the HMD 200 or may be detected by the camera 214 in front of the HMD 200. The control input sensor 217 to detect gesture can include one or more cameras, infrared sensors, motion sensors, or the like. Gesture controls can include any type of hand or finger motion, including but not limited to: pinching, pointing, swiping, circling, grasping, twisting, or the like. When detecting eye-based commands, the control input sensor 217 is able to sense the user's eye position, motion, dwell time (stare), gaze and the like, for purposes of determining the user's intended command and controlling the HMD 200, HMD controller 210, navigation controller 26, and/or manipulator controller 54 accordingly. The eye-based commands may be detected using an eye-tracker that is positioned to face the user's eyes, e.g., in front of the HMD display 208. Eye-based controls can include any type of eye-command, including but not limited to: selecting an object, moving an object, or the like. In one example, the user can select a computer-generated object displayed by the HMD 200 by staring at the object for a threshold amount of time. The HMD can also control input sensors 217 in the form of a microphone for recording verbal commands. The HMD controller 210 can process the verbal commands and control the HMD display 208 in response.

Any of the described components of the HMD 200 that can sense information or process sensed information (including but not limited to, the HMD controller 210, the video camera 214, tracking sensors 212, IMU 216, and/or control input sensors 217) can be understood as being part of a “sensing system” of the HMD 220. The sensing system is identified by numeral 219 in FIG. 2.

The HMD 200 can be registered to one or more objects used in the operating room, such as the tissue being treated, the surgical tool 22, the manipulator 12, the trackers 46, 48, the localizer 34, and/or the like. In one implementation, a local coordinate system is associated with the HMD 200 to move with the HMD 200 so that the HMD 200 is fixed in a known position and orientation in the HMD coordinate system. The HMD 200 can utilize the tracking sensors 212 to map the surroundings and establish the HMD coordinate system. The HMD 200 can then utilize the camera 214 to find objects in the HMD coordinate system. In some implementations, the HMD 200 uses the camera 214 to capture video images of markers attached to the objects and then determines the location of the markers in the local coordinate system of the HMD 200 using motion tracking techniques and then converts (transforms) those coordinates to the HMD coordinate system.

In another implementation, a separate HMD tracker 218 (see FIG. 1), similar to or different from the trackers 46, 48, could be mounted to the HMD 200 (e.g., fixed to the support structure 202). The HMD tracker 218 can have its own HMD tracker coordinate system that is in a known position/orientation relative to the local coordinate system of the HMD 200. Alternatively, the tracker coordinate system could be calibrated to the local coordinate system using calibration techniques. In this implementation, the local coordinate system becomes the HMD coordinate system. The localizer 34 could then be used to track movement of the HMD 200 via the HMD tracker 218 and transformations could then easily be calculated to transform coordinates in the local coordinate system to the localizer coordinate system, the anatomy coordinate system, the manipulator coordinate system, or other coordinate system.

A registration device may be provided with a plurality of registration markers and/or localizer markers to facilitate registering the HMD 200 to the localizer coordinate system. The localizer marker positions can be known or determined relative to the registration markers. For example, a structure can include the localization and registration markers in a fixed relationship. The HMD 200 locates the registration markers on the registration device in the HMD coordinate system via the camera 214 thereby allowing the HMD controller 210 to create a transform from the registration coordinate system to the HMD coordinate system. The localizer 34 determines the location of the localization markers in the localizer coordinate system. The HMD controller 210 then determines where the localizer coordinate system is with respect to the HMD coordinate system so that the HMD controller 210 can generate images having a relationship to objects in the localizer coordinate system or other coordinate system. The registration device or any technique for registering and/or calibrating the HMD 200 to another coordinate system can be like that described in U.S. Pat. No. 10,499,997, entitled “Systems and Methods for Surgical Navigation”, the entire contents of which are hereby incorporated by reference in their entirety.

During use, for example, the localizer 34 and/or the navigation controller 26 can send data on an object (e.g., the cut volume model) to the HMD 200 so that the HMD 200 knows where the object is in the HMD coordinate system and can display an appropriate content in the HMD coordinate system. Once registration is complete, then the HMD 200 can be used to visualize computer-generated content in desired locations with respect to any objects in the operating room. Although certain transforms have been described, it is understood that the HMD 200 can operate without requiring any such transforms. The HMD 200 can display content without registering to the bone, or any part of the surgical system 10.

### III. Surgical Tracker Device/System Utilizing a Digital Display Screen

Described herein are various implementations of improved tracker devices TD, tracker systems TRKS, software or non-transitory computer readable mediums, or methods that utilize and involve a digital display screen DS for presenting diverse types of information or graphics. Examples of various trackers or tracker devices have been described above using reference numerals 46, 48. This section provides additional implementations of the trackers that can be used with any aspect of the surgical system 10 described above. While the trackers described in this section include features not described above, it is understood that any of the trackers described herein may include some or all of the features of the trackers or tracking systems described above. For simplicity, the tracker system in this section is referred to by reference numeral TRKS and the tracker device is referred to by reference numeral TD.

### 1. Example System/Hardware Configurations on Tracker System/Device

The tracker system TRKS comprises the tracker device TD and at least one controller. The tracker device TD is a physical object that is configured to attach or be attached to an object to be tracked. When attached to the object, the tracker device TD can move with the object. One or more controllers process information or signals and controls the tracker device TD. The one or more controllers can be remote from the tracker device TD or integrated with the tracker device TD. When integrated with the tracker device TD, the controller can be a tracker controller TC, as shown in FIG. 2. The tracker controller TC can be coupled to non-transitory storage mediums and one or more processors for executing instructions that can be stored in memory. As shown in FIG. 2, the tracker device TD comprises a housing H which can house any of the components of the tracker device TD described herein, including the tracker controller TC.

Additionally, or alternatively, when remote from the tracker device TD, the one or more controllers can be any suitable controller, such as the navigation controller 26, the camera controller 42, manipulator controller 54, tool controller, and/or HMD controller 210. Any of these controllers TC, 26, 42, 54, 210 can be utilized independently or in combination for performing any of the capabilities described herein. Furthermore, as the tracker system TRKS comprises a controller, the tracker system TRKS can include any one or more of the described controllers TC, 26, 42, 54, 210. The various controllers TC, 26, 42, 54, 210 are configured to communicate with each other wirelessly (e.g., through Wi-Fi, Bluetooth, or any other wireless connection) or through wired connections (e.g., ethernet, etc.) and can be equipped with any appropriate hardware to enable such communications.

Because the difference between the tracker device TD and tracker system TRKS depends on the location of the one or more controllers TC, 26, 42, 54, 210, it should be understood that tracker device TD and tracker system TRKS may be interchangeable for any of the following description. Hence, any components, features, or capabilities described herein with reference to the tracker device TD can be fully performed by the tracker system TRKS.

The tracker device TD is detectable by any one or more tracking system that can detect graphics presented by the digital display screen DS. Examples of such tracking systems include but are not limited to: optical tracking systems, such as the localizer 34 (or camera) of the navigation system 20, the HMD 200 and camera 214 of the HMD 200, a camera coupled to a surgical tool 22, (such as an endoscope, a laparoscopic, arthroscope, hand-held robotic tool, etc.), a camera coupled to the manipulator 14, or a camera of a second tracker device TD (which will be described below). For simplicity in description, any one or more of these tracking systems will be referred to as the tracking system TRKSYS. One tracker device TD may be simultaneously detectable by multiple tracking systems TRKSYS. Multiple tracker devices TD may be simultaneously detectable by one or more tracking systems TRKSYS.

The tracker device TD can be coupled to any object to be tracked, which can be any of those described above and shown throughout the Figures, such as, but not limited to: any patient anatomy (e.g., femur F or tibia T), any surgical tool 22 (such as a probe, hand-held robotic tool, drill, screwdriver, implant inserter, retractor, limb holder, tool holder), a robotic manipulator 14, a tool or end effector attached to the manipulator 14, a base of the manipulator 14, a link of the manipulator 14, an imaging system (such as a C-arm or ultrasound scanning tool), a camera tool (such as an endoscope, laparoscope, arthroscope, or microscope), the HMD 200, a surgical table, or the like.

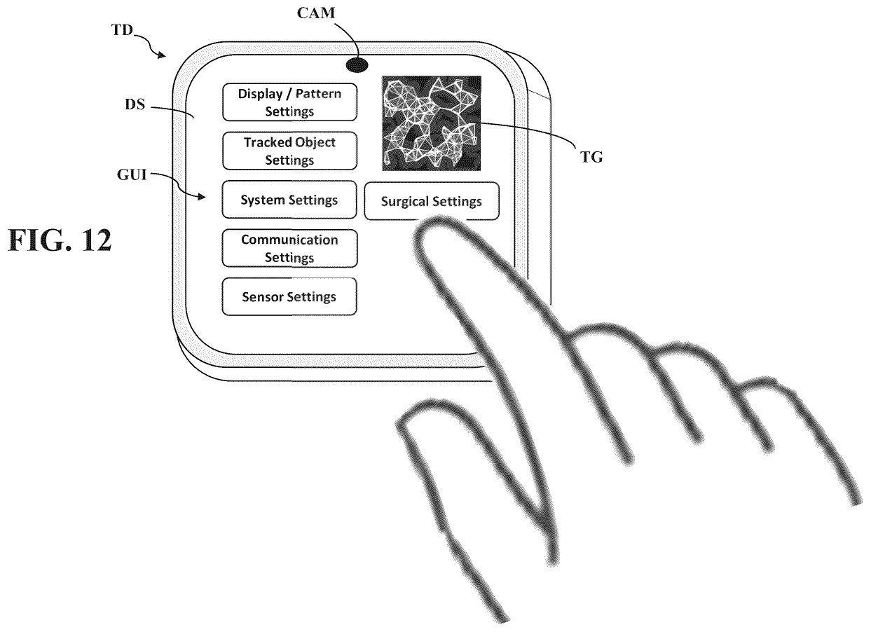

As shown throughout the various Figures, the tracker device TD comprises a digital display screen DS. The digital display screen DS is configured to present various types of content and/or graphics, as will be described below. The digital display screen DS can be supported by the housing H of the tracker device TD. The digital display screen DS can have any resolution sufficient to meaningfully convey digital content and/or graphics. In one example, the digital display screen DS has a resolution of at least 200 pixels by 200 pixels, and up to 4K or higher resolution. As shown in FIG. 12, the digital display screen DS can be a touch-screen controllable display such that a user can interact and control aspects of the display screen DS or tracker device TD. For example, the touch screen can be enabled by capacitive or infrared-based technology and can be a layer of the digital display screen DS. The digital display screen DS can by any type of digital display, such as any type of LED, LCD, OLED, micro-OLED, Quantum LED (QNED, QLED), micro-LED, mini-LED, dot matrix LED, RGB display, or the like.

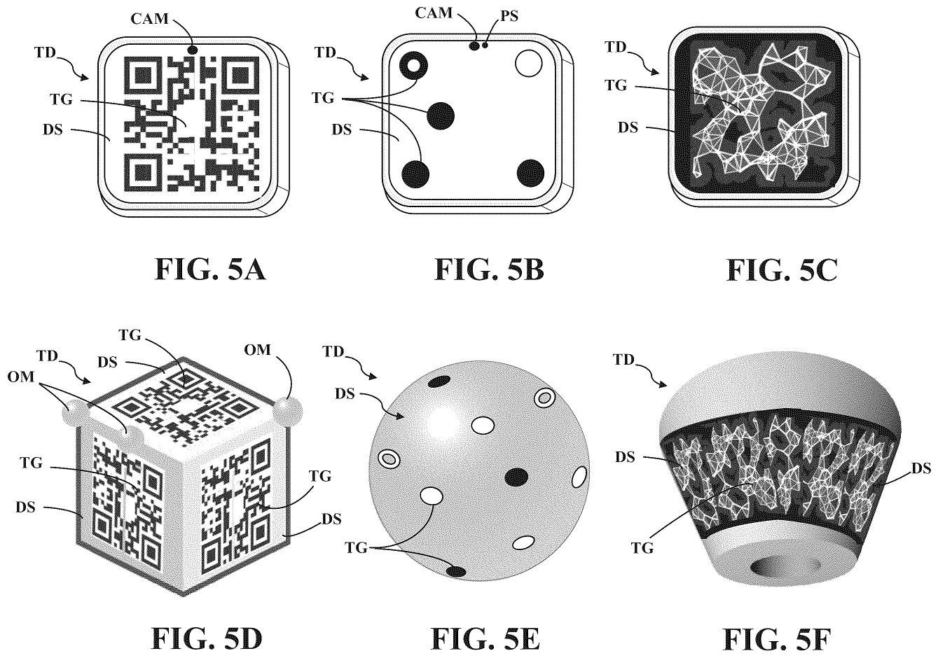

In some examples, such as those shown in FIGS. 5A, 5B, and 5C, the digital display screen DS can have a flat configuration. In other examples, the digital display screen DS can have a curved, contoured, irregular, and/or rounded configuration. For example, in FIG. 5E, the digital display screen DS has a spherical configuration. In FIG. 5F, the digital display screen DS has an irregular (e.g., bulb) shaped configuration. The digital display screen DS can have other types of configurations, including but not limited to a semi or partially spherical configuration, a semi or partially cylindrical configuration, or the like.

The tracker device TD and digital display screen DS can include any suitable shape and size. The digital display screen DS may occupy all, substantially all, or part of any one or more faces of the housing H of the tracker device TD. In some instances, the tracker device can include multiple digital display screen DS that can face in different directions from one another. For example, the housing H can include multiple faces with a digital display screen DS provided on any number of such faces. As shown in FIG. 5D, the housing H of the tracker device TD includes a cube-shape and a digital display screen DS can be provided on each face of the cube. Such multi-screen configurations can be implemented for any 3D geometric shape of the housing H. For instance, any of the flat tracker devices TD in FIGS. 5A-5C, can also include a digital display screen DS on the opposing backside of the tracker device TD. When the digital display screen DS is a (partial or full) sphere or cylinder or curved surface, the digital display screen DS can span up to 360 degrees to provide a viewing angle from any perspective. For example, the digital display screen DS could be shaped as quarter, half, or full ring that is wrapped about one of the links 58 of the manipulator 12. The digital display screen DS can be readily visible for any given pose or movement of the manipulator 12 during a procedure, The digital display screen DS could itself, from the housing H of the tracker device TD.

In one implementation, to provide a lightweight, non-obstructing design for attachment to objects, the tracker device TD and digital display screen DS can have a compact configuration. For example, the tracker device TD may be easily grasped by the hand of a user. The tracker device TD can have any suitable dimension. For example, the tracker device TD in FIG. 5A can have a volume (length, width, depth) of 50×50×10 mm. When a larger tracker device TD is needed, such as the one attached to the base of the manipulator 14 in FIG. 1, the tracker device TD may have a relatively larger dimensions (e.g., 100×100×10 mm). Such dimensions are provided only as examples, and the figures may not represent this exact scale. The dimension of the tracker device TD or digital display screen DS may be sized as needed for the appropriate application or object to be tracked.

In another example, the tracker device TD is a portable electronic device (such as a smartphone, tablet, etc.) that has capabilities beyond being used as a tracker device TD. Such supplemental capabilities include personal use, such as the capability for cellular communication, storing photos, or any other feature provided by smartphones and tablets. In some cases, the portable electronic device can be a folding device, such as a paper-thin folding device. In other implementations, the tracker device TD is specifically designed solely for the purposes of tracking objects in a surgical environment.

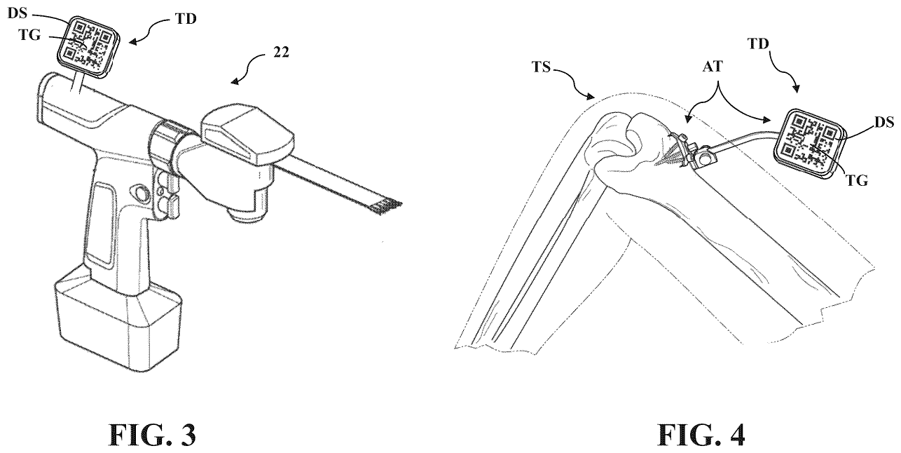

The tracker device TD can be coupled to the object to be tracked according to various manners. In one implementation, the tracker device TD can be integrated into the object to be tracked. With reference to FIG. 3, one example is illustrated of a surgical tool 22, i.e., a hand-held saw tool, which has the tracker device TD integrated into the body of the tool 22. Such integration can be within the body of the object or extending from the body of the object. Such integration is contemplated for any of the objects to be tracked.

In another implementation, the tracker device TD can be releasably attached to the object to be tracked. Such releasable attachment is contemplated for any of the objects to be tracked. With reference to FIG. 4, one example is illustrated whereby the tracker device TD has been attached to the target site TS or patient anatomy using an attachment AT. In this configuration, the tracker device TD can be coupled to the attachment AT and the target site TS in preparation for surgery and removed from the attachment AT and target site TS after use. In FIG. 4, the attachment AT comprises a bone plate that is secured to the femur using screws. The attachment AT can be a kinematic attachment. For example, the bone plate comprises kinematic balls that connect to a receiver attached to a stem with kinematic surfaces inside a cavity of the receiver. When attached, the kinematic balls and receiver form a kinematic connection to repeatedly secure the components in six degrees of freedom. The tracker device TD is releasably attached to the opposing end of the stem. This example attachment AT can be like that described in U.S. Pat. No. 10,537,395, entitled “Navigation Tracker with Kinematic Connector Assembly”, the entire contents of which are hereby incorporated by reference. As such, any components of the attachment AT can be directly coupled to the housing H of the tracker device TD. When coupled to the manipulator 12, the attachment AT can be a housing/cavity with electrical terminals, wherein the housing/cavity is formed into base, link or end effector of the manipulator 12. In such cases, the tracker device TD can have corresponding electrical terminals, and the tracker device TD can be plugged into the housing/cavity to secure the tracker TD and provide the requisite electrical connections. Although FIG. 4 shows on example of how the tracker device TD can be releasably attached to the object to be tracked, there are numerous other ways, including the tracker device TD being clamped, pinned, mounted, fastened, or secured to the surgical object in any manner. For example, the tracker device TD could be secured to an elastic band, bendable strip that can be secured around the object to be tracked (such as a limb or robotic link). In another example, when the tracker device TD is a portable electronic device, the attachment AT can be a rigid or adjustable device holder sized for clamping to or slidably receiving the portable electronic device.

Other example attachments can be like those described in: US Patent App. Pub. No. 2023/0338093, entitled “Sterilizable Surgical Device With Battery Switch”, US Patent App. Pub. No. 2022/0257334, entitled “Clamp Assembly For Fixing A Navigation Tracker To A Portion Of Bone”, U.S. patent application Ser. No. 18/392,014, entitled “Surgical Clamp Assembly For Fixing A Navigation Tracker To A Portion Of Bone”, U.S. Provisional Patent App. No. 63/526,733, entitled “Surgical Tracker Assemblies With Optimized Size, Weight, And Accuracy”, and/or U.S. Provisional Patent App. No. 63/472,138, entitled “Assembly And Method For Mounting A Tracker To Bone”, the entire contents of these disclosures being hereby incorporated by reference.

Additional components of the tracker system TRKS and tracker device TD will now be described with reference to FIG. 2. These components can be supported by or within the housing H of the tracker device TD. Capabilities and functionality of these components can involve or be implemented by any one or more of the controllers TC, 26, 42, 54, 210.

In addition to the digital display screen DS, the tracker device TD may optionally also comprise one or more optical markers OM (e.g., as shown in FIGS. 5D and 11). The optical markers OM can be like those described above with respect to trackers 46, 48. The optical markers OM are physical objects (not presented on the display screen DS) that may be coupled or attachable to the housing H of the tracker device TD. The optical markers OM may include passive markers. For example, the tracker device TD can have at least three passive tracking elements or markers (e.g., reflectors) for transmitting light signals (e.g., reflecting light emitted from the tracking system TRKSYS). In other implementations, the optical markers OM may include active tracking markers. The active markers can be, for example, light emitting diodes transmitting light, such as infrared light. Active markers may be controlled by the tracker controller TC. Active and passive arrangements are possible. The one or more optical markers OM can be used to provide communication with or supplemental tracking information to the tracking system SYSTRK. For example, such optical markers OM may be used to detect the tracker device TD in situations where the digital display screen DS not yet initialized or otherwise inactive. Optical markers OM can also be used to provide a supplemental form of tracking (concurrent with detection of the digital display screen DS) for tracking redundancy and providing additional confidence in tracking. In other examples, due to the fixed relationship between the optical markers OM and the digital display screen DS, the tracker device TD may operate as a registration device to facilitate registration of the HMD 200 and the localizer coordinate system. For example, the localizer 34 may detect the optical markers OM and the HMD 200 may detect the trackable graphic TG. Using the fixed relationship, the HMD 200 and localizer 34 can be registered to one another.

The tracker device TD may include one or more time-of-flight sensors TFS. The time-of-flight sensor TFS can be a range-finder, laser, LIDAR, or LED enabled sensor. The time-of-flight sensor TFS can also be embodied as by a camera CAM on the tracker device TD. Based on the time difference between the emission of the light and its return to the time-of-flight sensor TFS after being reflected by an object, the time-of-flight sensor TFS and/or tracker controller TC is able to measure the distance between the object and the time-of-flight sensor TFS. Also, based on the difference between the time the light is projected on the tracker device TD to the time it is detected by the time-of-flight sensor TFS, the controller TC is able to determine the position of the tracking device TD. As will be described below, the one or more controllers TC, 26, 42, 54, 210 are configured to utilize measurements from the time-of-flight sensor TFS to determine the relative spatial relationship between the tracker device TD and the tracking system TRKSYS. Additionally, or alternatively, the time-of-flight sensor TFS can be employed by the tracking system TRKSYS. The tracker device TD can include multiple time-of-flight sensors TFS, e.g., one for each side including a digital display screen DS, or on different sides of the housing H.

The tracker device TD can include transmitter or receiver (or transceiver) (TX) that can be utilized to communicate to or from a transceiver of the tracking system TRKSYS. Such communication may be, for example, infrared communication. Communication can be performed for various purposes, such as to enable the tracking system TRKSYS to initialize the tracker device TD. Other types of communication are contemplated that may configure how the tracker device TD operates, including power settings, display settings, communication settings, authentication, sensor settings, and the like. The transceiver TX can also utilize radio frequency transmission, e.g., to communicate higher-bandwidth data, such as inertial data, and the like. Examples of IR and/or RF transceivers that can be used by the tracker device TD and tracking system TRKSYS can be like those described in U.S. Pat. No. 10,555,781, entitled “High bandwidth and low latency hybrid communication techniques for a navigation system”, the entire contents of which are hereby incorporated by reference.

The tracker device TD can include a proximity sensor PS. The proximity sensor PS can be utilized to perform various capabilities for the tracker device TD. For example, the proximity sensor PS can detect absence of environmental activity and in response place the tracker device TD or digital display screen DS in a sleep mode to conserve energy. In another example, the proximity sensor PS can detect presence of environmental activity to ensure the tracker device TD or digital display screen DS remain active. The proximity sensor PS can also function as an ambient light sensor. The proximity sensor PS can be built into the digital display screen DS (as shown in FIG. 5B) or can be located elsewhere on the housing H. In one example, the proximity sensor PS includes an infrared LED light and an IR light sensor to detect proximate activity. The tracker device TD can include multiple proximity sensors PS, e.g., one for each side including a digital display screen DS, or on different sides of the housing H.

The tracker device TD can also include one or more inertial sensors IMU. The IMU can be a gyroscope, accelerometer, magnetometer, MEMS device, or the like. Measurements from the inertial sensor IMU can be utilized to perform any one or more of the following: change an orientation of text/graphics presented on the digital display screen DS; communicate the measurements to the tracking system TRKSYS to supplement tracking of the tracker device TD; detect that the tracker device TD is being moved by a user; and/or detect an undesired motion of the tracker device TD.

The tracker device TD can include one or more cameras CAM. The camera CAM is configured to capture image or video data in the environment of the tracker device TD. The image or video data can be of any object, including to detect the object to be tracked or another tracker device TD. The image or video data can be utilized to perform one or more of the following: detect an event; modify what is presented on the digital display screen DS; detect presence or absence of any surgical object; detect presence or absence of the tracking system TRKSYS; present the image or video data on the digital display screen DS; and/or detect a face of a user to authenticate use of the tracker device TD. By including a camera CAM, the tracker device TD can perform any of the capabilities described by any of the described tracking system TRKSYS, including tracking the pose of objects, detecting surgical information or events, or the like. The camera CAM can be built into the digital display screen DS (as shown in FIG. 5B) or can be located elsewhere on the housing H. The camera CAM can include a compact design to reduce size/weight of the tracker device TD. The camera CAM can be any suitable camera, such as a 12 MP or higher camera with any appropriate focal length or zooming capability. The tracker device TD can include multiple cameras CAM, e.g., one for each side including a digital display screen DS, or on different sides of the housing H.

The tracker device TD include a power source PWR. The power source PWR provides energy to power the digital display screen DS and various sensors of the tracker device TD. The power source PWR can be a battery, such as a lithium-ion battery, alkaline battery, or the like. The power source can be rechargeable. The tracker device TD can include a charging port (e.g., USB-C connection) for enabling recharging of the power source. In some cases, the power source can be an energy harvesting power source that generates energy from motion of the tracker device TD or other sources and stores the power for use without requiring any external power source or recharging. The power source can be built into the housing H or removable from the housing H. The power source can be a battery switch, which fits into a cavity in the housing H. When the battery switch is rotated relative to the cavity, the battery switch is sealed to the housing H and makes electrical contact to power the tracker device TD. The battery switch may be disposable. The power source or battery can be like those described in: US Patent App. Pub. No. 2023/0338093, entitled “Sterilizable Surgical Device With Battery Switch”, the entire contents of which are hereby incorporated by reference. In other cases, the tracker device TD may be wired to an external power source PWR.

In one example, the tracker device TD is sterilizable and configured to be used in the sterile field. In one instance, the housing H may be hermetically sealed from the elements. The display screen DS may also be hermetically sealed, or placed under a sealed transparent layer that is also hermetically sealed to the housing H and thermally protected. Certain components can be removed from the tracker device TD to facilitate sterilization. In one example, the digital display screen DS and the tracker controller TC can be removed from the housing H, and the housing H can be sterilized. In another example, the housing H can include cavity disposed therein for the tracker controller TC. The cavity can open to the exterior of the housing H through at least one channel formed in the housing H. The tracker controller TC (which can comprise PCB) can be coated with a heat-resistive coating, such as a Parylene coating. The entire tracker device TD can be sterilized and the sterilization fluid can enter the cavity and drain from the channels during sterilization. To facilitate this capability, the tracker device TD can employ the configuration and features of those described in: US Patent App. Pub. No. 2023/0338093, entitled “Sterilizable Surgical Device With Battery Switch”, the entire contents of which are hereby incorporated by reference.

In another example, the tracker device TD is configured to interface with a surgical or sterile drape that prevents exposure of the tracker device TD with the sterile field. In one example, the drape can include a transparent window configured to cover the digital display screen DS while enabling the contents of the display screen DS to be visible. The transparent window can be coupled or configured to couple to a drape or the housing H of the tracker device TD. A flexible drape material can extend from the transparent window to wrap around the tracker device TD. In another example, the housing H of the tracker device TD can support a drape attachment mechanism that enables attachment of the drape to the tracker device TD. For example, the housing H can define a channel that receives an elastic member (such as a flexible band) to secure the drape over the digital display screen DS. In other examples, a sterile cover, sticker, or sheet sized for the housing H or digital display screen DS can be installed. When the tracker device TD is attached to the manipulator 12, the drape can be for the manipulator 12 and the tracker device TD can present information or graphics through the manipulator drape using any of the described techniques. Any of the above implementations may be utilized in part, or in whole. Example techniques by which the tracker device TD can interface with a drape, while still providing its functionality can be like those described in U.S. Pat. No. 9,713,498, entitled “Assembly For Positioning A Sterile Surgical Drape Relative To Optical Position Sensors”, the entire contents of which are hereby incorporated by reference.

### 2. Presentation of Computer-Generated Trackable Graphic(s) on Digital Display Screen of Tracker Device

As introduced above, the tracker device TD is configured to present one or more computer-generated trackable graphics (hereinafter ‘trackable graphic TG’) one or more digital display screens DS. By doing so, the tracker device TD can be made detectable by the tracking system TRKSYS detecting the trackable graphic TG to facilitate tracking of a pose of the surgical object to which the tracker device TD is attached. In one implementation, the tracker controller TC implements control and instructions to present the trackable graphic TG on the digital display screen DS. However, as described, control and presentation of the trackable graphic TG on the digital display screen DS can additionally or alternatively be implemented by any other controller(s) 26, 42, 54, 210. The trackable graphic TG can be remotely detected by any tracking system TRKSYS or camera source described herein, such as by an optical tracking systems, such as the localizer 34 (or camera) of the navigation system 20, the HMD 200 and camera 214 of the HMD 200, a camera coupled to a surgical tool 22, (such as an endoscope, a laparoscopic, arthroscope, hand-held robotic tool, etc.), a camera coupled to the manipulator 14, or a camera CAM of a second tracker device TD. The trackable graphic TG can be presented in a manner configured to enable the tracking system TRKSYS to determine the pose of the surgical object in at least five-degrees of freedom, and up to six degrees of freedom.

The trackable graphic TG can be presented on the digital display screen DS according to numerous manners and forms. For example, one digital display screen DS can present one or multiple trackable graphics TG at the same time or separate times. Multiple digital display screens DS can present the same trackable graphic TG. Multiple digital display screens DS can present different trackable graphics TG. The trackable graphic TG can occupy an entirety or a portion of any digital display screen DS area. The digital display screen DS can present any appropriate background or surround in conjunction with the trackable graphic TG, e.g., to make the trackable graphic TG more visible. For example, the background can be white/black or can be updated based on detected environmental or ambient conditions. One trackable graphic TG can span, extend across, or move across multiple digital display screens DS. When the digital display screen DS is curved, the trackable graphic TG can be presented in a curved manner on the digital display screen DS and can move about the curved digital display screen DS. A configuration or geometry of the trackable graphic TG may be configured to change, e.g., during use of the tracker device TD. One digital display screen DS may toggle on/off any trackable graphic TG. Any one or more trackable graphics TG may be swapped for presentation between one digital display screen DS and another digital display screen DS. The trackable graphic TG may be static or may change pose on one or more digital display screens DS. The trackable graphic TG may change size or shape on one or more digital display screens DS. The trackable graphic TG may move about one or more digital display screens DS. The trackable graphic TG may change from on one or more digital display screens DS. The trackable graphic TG can be optimized for the respective tracking system TRKSYS detecting the tracker device TD. Such optimization can include any of the described implementations explaining when, where and how to configure and/or display the trackable graphic TG on the digital display screen DS. In one example, the intensity or frequency of the light emitted by the digital display screen DS may change. Light intensity or frequency may be changed, for example, to be optimized for the tracking system TRKSYS the tracker device TD is working with. Given the various lighting systems in the operating room, the intensity or frequency and display graphics settings could be optimized for every procedure, for every location of the device, or based on changing lighting conditions in the surrounding area at different times during a procedure. In other examples, the tracker device TD can present multiple trackable graphic TG that can be detected by different tracking systems TRKSYS. For example, one portion of the display screen DS can present a first trackable graphic TG for a first tracking system TRKSYS and a second portion of the display screen DS can present a second trackable graphic TG for a second tracking system TRKSYS. The trackable graphics TG can be presented side-by-side, on different sides, or overlapping one another. In another example, trackable graphics TG can be displayed in a pulsating or strobing manner, whereby the display screen DS alternates presentation of different trackable graphics TG on the same region of the display screen DS. This pulsating or strobing can be at any frequency, such as at 60 Hz or greater. Any of these manners of controlling the trackable graphic TG can be utilized individually or in combination.

As will be described below, the user may utilize a graphical user interface GUI implemented by the tracker device TD to control settings to specify when, how, and where the trackable graphic(s) TG, or any other content or information is/are presented on the digital display screen(s) DS.

FIGS. 5A-5F illustrate numerous examples of the trackable graphic TG. In FIGS. 5A and 5D, the trackable graphic TG comprises a QR code. The QR code can be any type of QR code, such as a Model 1 QR code, Micro QR code, iQR code, Secure Quick Response (SQR) code, Frame QR code, or a High Capacity Colored 2-Dimensional (HCC2D) code, or the like. In some cases, the QR code can be a dynamic QR code encoded with supplemental information or instructions for the tracking system TRKSYS. The one or more controllers TC, 26, 42, 54, 210 can repeatedly change the QR code or dynamic QR code during tracking. For example, the size, shape, configuration, intensity, density, and/or color of the codes can be changed. In one implementation, the QR code is implemented as a particle cloud of points that dynamically change shape and/or color. FIG. 5A shows a QR code on a flat digital display screen DS of the tracker device TD. FIG. 5D shows multiple QR codes on multiple flat digital display screen DS arranged on various sides of the cube-shaped tracker device TD. As described, the multiple codes can be the same or different from one another. Of course, one or more QR codes can be presented in various manners depending on the configuration of the tracker device TD, digital display screen(s) DS, and/or conditions affecting presentation.

In another example, FIGS. 5B and 5E illustrate the trackable graphic TG as digital fiducials or markers. These digital fiducials or markers are digitally presented on the digital display screen DS, and hence, are different from the physical optical markers OM, shown in FIG. 5D, for example. The digital fiducials may, but need not, be infrared detectable fiducials. The digital fiducials can be presented in any number, style, and arrangement. The digital fiducials can be various sizes, colors, and shapes. The one or more controllers TC, 26, 42, 54, 210 can repeatedly change the features of the digital fiducials during tracking. For example, the size, shape, configuration, intensity, density, and/or color of the digital fiducials can be changed. FIG. 5B shows digital fiducials on a flat digital display screen DS of the tracker device TD. At least three digital fiducials are presented, which can help the tracking system TRACKSYS detect the pose of the tracker device TD in at least five degrees of freedom. The digital fiducials are shown as circles or dots, with different filling or outlining. A curved or multi-faced digital display screen DS with digital fiducials may further improve tracking accuracy. For example, FIG. 5E shows multiple digital fiducials on a spherical digital display screen DS. The digital fiducials may be static or may move about the spherical display screen DS. If a cube shaped tracker device TD is used (e.g., such as FIG. 5D), the multiple display screen DS may each show the same digital fiducial arrangement or a different digital fiducial arrangement. In another example, each digital display screen DS may show one or more digital fiducials, which when viewed collectively among the display screens DS, form the arrangement of digital fiducials. Of course, digital fiducials can be presented in various other manners depending on the configuration of the tracker device TD, digital display screen(s) DS, and/or conditions affecting presentation.

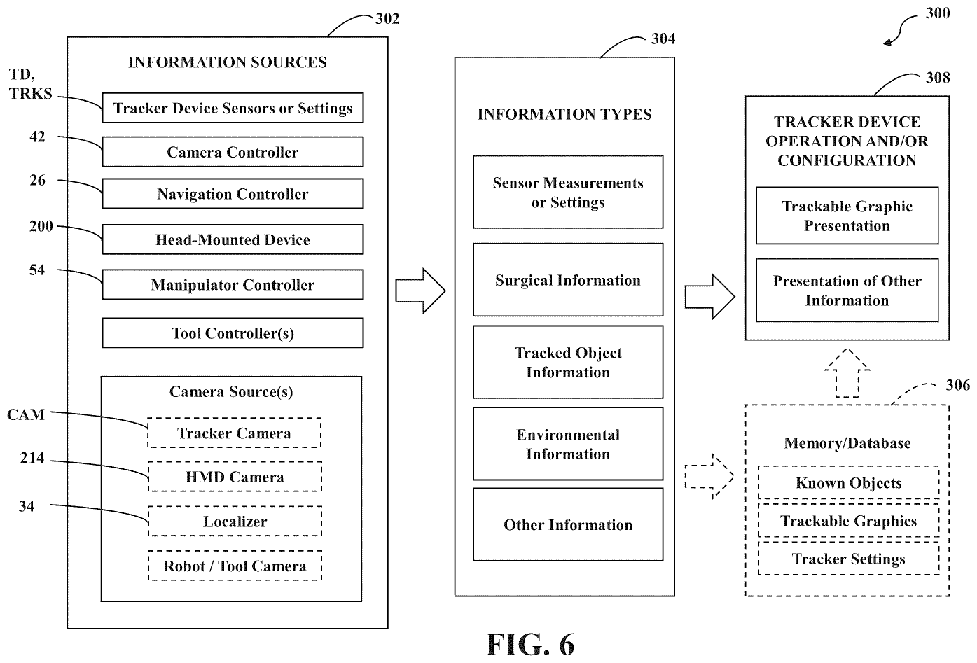

In one implementation, the digital fiducials can be arranged or presented on the digital display screen DS to mimic predetermined geometries of physical optical markers OM for other tracking devices. For example, as will be described with respect to FIG. 6, the one or more controllers TC, 26, 42, 54, 210 can obtain information and look up, in a memory or database, predetermined geometries of physical optical markers OM for other tracking devices. From this obtained information, the one or more controllers TC, 26, 42, 54, 210 can transform the predetermined geometry of physical optical markers OM into the geometry and arrangement of the digital fiducials on the digital display screen DS.

FIGS. 5C and 5F illustrate other examples wherein the trackable graphics TG are implemented as static or dynamically changing point clouds. FIG. 5F shows the point cloud displayed 360 degrees about the display screen DS of a bulb-shaped tracker device TD. Other examples of trackable graphics TG are contemplated, including but not limited to any bar codes, icons, graphics, text, images, video, or any predefined or irregular arrangement of points, lines, planes, or geometry that can be detected by the tracking system TRKSYS to track a pose of the tracker device TD.

### 3. Tracker Device Configuration/Operation Inputs

FIG. 6 illustrates an example method 300 illustrating one or many ways to operate and/or configure the tracker device TD or tracker system TRKS. For example, the described information/inputs may affect what, when, where, and how the trackable graphic TG or any other information presented by the digital display screen DS can be generated, presented, or modified. These described techniques may be optional and the tracker device TD may be pre-configured without requiring additional input. The techniques described in FIG. 6 may be performed pre-operatively or intra-operatively. Any of these techniques may cause automatic configuration or operational changes to the tracker device TD, e.g., without user input. In other scenarios, the user may provide input configuration or operational changes to the tracker device TD.

At 302, information is provided from one of a plurality of information sources, which can provide information affecting how the tracker device TD or tracker system TRKS may operate or be configured. The sources of information include any of the described components of the system 10 and other sources, such as but not limited to sensors or settings: from the tracker device TD/tracker system TRKS, the camera controller 42, the navigation controller 26, the HMD 200, the manipulator controller 54, the tool controller, or any other camera source, such as the camera CAM of the tracker device TD, a camera 214 of the HMD 200, a camera attached to any surgical tool or device 22, or a camera attached to the manipulator 14. Other sources of information are contemplated beyond those described.

At 304, information is provided from any one or more of the information sources. The information can be of diverse types depending on the source. For example, the information from any one or more of the described information sources can include but are not limited to: sensor measurements or settings; surgical information; tracked object information; environmental information; or any other type of information. Any of these information types can be utilized individually or in combination, and the information can be obtained from any one or more of information source and at any given time.

i. Information from Tracker Device

Information affecting how the tracker device TD or tracker system TRKS may operate or be configured can be from the tracker device TD or tracker system TRKS itself. For example, the tracker device TD or tracker system TRKSYS can utilize measurements or data from any of the described hardware, e.g., controller TC, display screen DS, time of flight sensor TFS, transceiver TX, proximity sensor PS, inertial sensor IMU, camera CAM, or power source PWR. Similarly, any measurements or data from the HMD 200 can be obtained, such as from the video camera 214, tracking sensors 212, display 208, IMU 216, or control inputs 217.

For example, information can be provided by the time-of-flight sensor TFS, can assist to determine a relative spatial relationship between the tracker device TD and the tracking system TRKSYS. This may be advantageous when the tracker device TD or tracking system TRKSYS is/are moving. The relative spatial relationship can be processed by the controller to determine when or how to display the information on the display screen DS. For instance, the relative spatial relationship may cause a change in presentation or pose of the trackable graphic TG. The location of the computer-generated trackable graphic TG on the digital display screen DS can be changed to react to a movement of the tracker device TD, for example, to react to acceleration or velocity changes. The tracker device TD may also communicate time-of-fight measurements to the tracking system TRKSYS to enable the tracking system to offset latency. In other examples, the trackable graphic TG can be encoded with time stamps to facilitate synchronization with the tracking system TRKSYS. The encoding can consider measurements from the time-of-flight sensor TFS.

In another implementation, the tracker device TD can obtain information about the tracking accuracy or tracking parameters of one or more of the various tracking systems TRKSYS. The accuracy or parameters, for example, can be defined relative to a primary viewing axis of the tracking system TRKSYS. Based on this obtained information, the tracking device TD may present a fixed trackable graphic TG optimized for tracking accuracy or tracking parameters of the tracking system TRKSYS. Additionally, or alternatively, dynamic trackable graphics TG could be used in combination with time-of-flight calculations to improve the tracking accuracy in any direction. The dynamic trackable graphics TG could be turned off after the fixed trackable graphic TG is optimized for the current tracking system TRKSYS and tracker device TD positions. These techniques address the deficiency of conventional static tracking devices that exhibit degradation in tracking accuracy in response to the tracking device not directly facing the respective tracking system.

The transceiver TX of the tracker device TD can receive information from the tracking system TRKSYS that may affect when or how information is displayed on the display screen DS. Such communication may be, for example, the tracking system TRKSYS sending a command initialize the tracker device TD and display the trackable graphic TG. Other types of communication are contemplated that may configure how the tracker device TD operates, including power settings, display settings, communication settings, authentication, sensor settings, and the like.

The proximity sensor PS of the tracker device TD may affect when or how information is displayed on the display screen DS. The proximity sensor PS can detect absence of environmental activity and in response place the tracker device TD or digital display screen DS in a sleep mode to conserve energy. In another example, the proximity sensor PS can detect presence of environmental activity to ensure the tracker device TD, digital display screen DS, or trackable graphic TG remain active. As an ambient light sensor, the proximity sensor PS can active the trackable graphic TG when the tracker device TD is moved or picked up. The proximity sensor PS may also detect a potential obstruction to the display screen DS. In other examples, the display screen DS could present a warning or notification based on proximity sensor PS measurements.

The inertial sensor IMU of the tracker device TD may affect when or how information is displayed on the display screen DS. For example, if the pose of the tracker device TD is changed, measurements from the inertial sensor IMU may change an orientation of text/graphics presented on the digital display screen DS (e.g., portrait mode to landscape mode). IMU measurements can change a configuration of the trackable graphic TG. For example, IMU measurements may detect that the tracker device TD is being moved by a user and/or detect an undesired motion of the tracker device TD. In turn, the trackable graphic TG can be adjusted to account for such movement or undesired movement. In other examples, the display screen DS could present a warning or notification regarding movement errors based on the IMU measurements.

The tracker device TD camera CAM can provide numerous sources of information that may affect presentation on the digital display screen DS. For example, the camera CAM can acquire image or video data. Such image or video can be of virtually anything in the operating room, including but not limited to, the surgical object being tracked, any surgical object, the target site, the tracking system TRKSYS, optical markers OM attached to any object, surgical actions (e.g., tool properties, operation, or movements, anatomy properties or movements, interaction between objects, and the like). In one case, the images or video data can be directly presented on the digital display screen DS. In another instance, the acquired images or video may be processed by any of the described controllers for deriving information, surgical information or surgical context from such images or video. The derived information or context can also be presented on the digital display screen DS, e.g., using notifications or graphics.

The camera CAM can detect presence, absence, or movement of any surgical object, including the object attached to the tracker device TD. In response a confirmation or warning message may be generated on the digital display screen DS. Presence, absence, or movement of the tracking system TRKSYS can be similarly detected for presenting relevant information on the display screen DS. For example, the trackable graphic TG may be presented when the tracking system TRKSYS is present, and the trackable graphic TG may not be presented when the tracking system TRKSYS is absent. In another example, the camera CAM may detect the type of tracking system TRKSYS, which can trigger presentation of a trackable graphic TG specifically adapted for the type of tracking system TRKSYS. The camera CAM can detect line-of-sight obstructions between the tracker device TD and the tracking system TRKSYS. This detected obstruction may cause movement of the trackable graphic TG to a new location one or more display screens DS or may cause display of a warning, error, or alert.

In some cases, the tracker device TD camera CAM can detect a second tracker device TD. For example, the second tracker device TD may present a trackable graphic TG that is detected by the camera CAM of the tracker device TD. The relative spatial relationship between the two tracker devices TD can be determined. This technique can be performed in a “daisy-chain” fashion among several tracker devices TD to determine more complex transforms or relationships between objects in the operating room.

The camera CAM can also detect a face of a user to authenticate use of the tracker device TD. Authentication may be implemented to ensure the tracker device TD is used by the specified surgeon or for a specific procedure or patient. Facial recognition software may be implemented by the controller TC to perform this task.

The power source PWR of the tracker device TD can also affect presentation on the display screen DS. For example, when a threshold level of low power is detected from the power source PWR, the controller TC can dim the display screen DS. When a threshold level of sufficient power is detected from the power source PWR, the controller TC can illuminate the display screen DS with full brightness. The display screen DS can also present information related to power level, such as percentage of battery life left (e.g., 50% battery remaining).

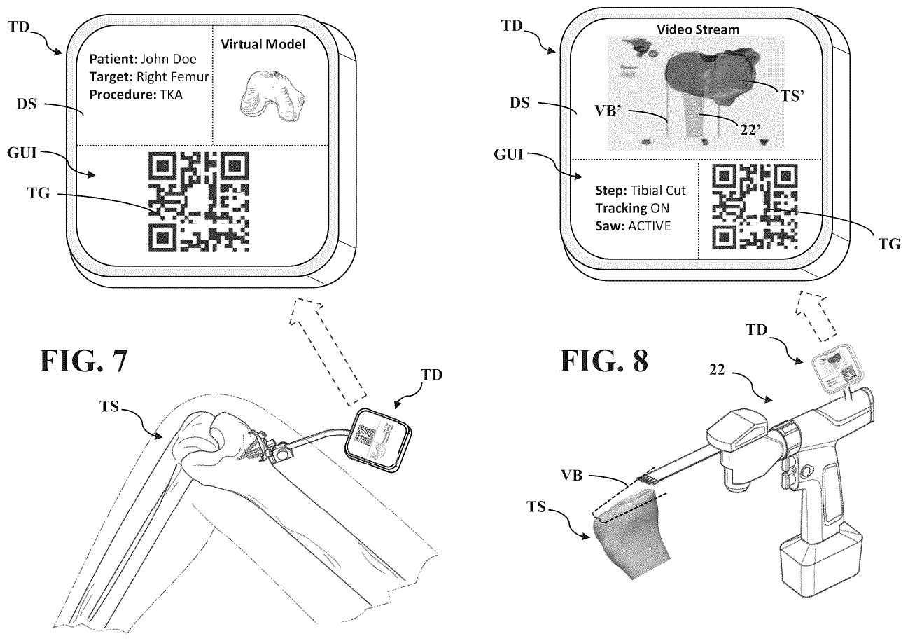

As shown in FIGS. 7, 8, 11 and 12, the user may utilize a graphical user interface GUI implemented by the tracker device TD to enable a user to provide input, for example, to modify settings or operation of the tracker device TD. For example, the GUI enables the user to control settings to specify what, when, how, and/or where information or trackable graphics TG is/are presented on the digital display screen(s) DS. The GUI may be interfaced with by a touch-screen display or any other type of user input. The GUI can provide various settings, including but not limited to display settings, pattern settings, tracked object settings, system settings, communication settings, sensor setting, or surgical settings. Regarding display/pattern settings, any of the described implementations of the trackable graphic TG can be configured using the GUI. For example, the size, shape, type, or location of the trackable graphic TG can be specifically set. The location can specify what specific display screen DS will present the trackable graphic. Any display settings of the trackable graphic TG can be modified, such as intensity, density, illumination, brightness, hue, color, strobing, resolution, display orientation, HDR setting, scaling, contrast, graphic position, focus, display format, idle timeout, or the like. Tracked object settings may define an identity or type the object being tracked by the tracker device TD, the type of connection or spatial relationship between the tracker device TD and the object being tracked, the operative side of the object being tracked, and the like. System settings can adjust any of the general operating settings of the tracker device TD, such as memory settings, sound settings, power settings, display settings, activation settings, communication settings, system information, notification settings, or the like. Communication settings can define parameters of communication between the tracker device TD and any other system. For example, communication settings could define whether or not the tracker device TD can communicate information to a remote server. Other settings, such as transceiver TX settings, IR/RF communication settings, can be specified. Sensor settings can change settings for any of the described sensors of the tracker device TD, such as operating parameters, sensitivity, or toggling such sensors on/off. Surgical settings can include any type of information or settings related to the surgical procedure, surgeon, or patient. For example, such settings may specify the type of procedure, a step of the procedure, surgical plan settings, patient parameters, the operative side of surgical object, surgical tool or manipulator settings, and the like. The GUI can also provide the ability to specify settings or configurations for the surgical object. For example, if the surgical object is the manipulator 12, the GUI can enable setting of the control mode, speed, virtual boundaries, surgical steps to be performed, or any other surgical settings. Any of these settings or configurations may be utilized independently or combination to directly configured when, where and how information or trackable graphics TG are displayed on the display screen DS. Additionally, any of these settings may be pre-set default or automatically configured settings for the tracker device TD, without requiring user interacting with the GUI.

ii. Information from External Sources

Information affecting how the tracker device TD or tracker system TRKS may operate or be configured can be from other sources external to the tracker device TD. For example, information provided by the navigation controller 26 and camera controller 42 may include tracking data, tracked pose information of any surgical object being tracked, including the tracker device TD, camera settings, spatial relationships or transforms between various objects, object geometry or tracker configurations, presence or absence of line-of-sight between the localizer and any tracked object, detection errors, tracking measurements, such as angle or distance of tracking between tracked object and localizer, the identification of objects being tracked, video stream data or images captured from optical sensors 40 of the navigation system, registration information, virtual boundary VB data, tool path data, surgical plan information, surgical step information, clinical application CA information (data, screens, video, images), notifications and warnings and the like.

Information provided by the manipulator controller 54 can include any robot data, including but not limited to a pose of the manipulator 14, a kinematic model of the manipulator 14, registration or calibration data related to the manipulator 14, robotic tool path or boundary data, robotic errors, robot settings, robotic operational data, force/torque sensor inputs, joint (torque) measurements, attached end effector 22 information, end effector 22 operational information, notifications, errors, and warnings, and the like.

Information provided by the tool controller can be any information related to any surgical tool 22 in the operating room, including hand-held tools or robotically controlled tools 22. Such information can be camera or video information if applicable (e.g., endoscope), tool settings, tool operating or control parameters, tool attachments, tool positional information, notifications and warnings, etc. When a camera is provided, such as any camera described herein (tracker camera CAM, HMD camera 214, localizer sensors 40, tool cameras, etc.), any information captured by such cameras can be utilized. The information may be video or image data captured by any one or more cameras. The video can be a live video stream, e.g., of a surgical environment or target site TS.

Any of these external sources can provide surgical information or surgical context to the tracker device TD. The surgical information can include but is not limited to: information about the surgical object, an identity of the surgical object, information about a surgical procedure or step of the surgical procedure, surgical plan information, surgeon preferences, virtual objects (e.g., bone models, tool models), medical imaging data, virtual boundary information, cut or respective volume information, clinical information, patient information (age, sex, BMI, bone density), deviations from or compliance to surgical workflows, detection of tool use or operation, tool inventory, implant information or inventory, planned trajectories, planned cut planes, planned implant positions, joint measurements, planned joint biomechanics, soft tissue or ligament information, live video feeds of the surgical site, images captured at the surgical site, a tracking status of the tracker device TD, an operation status of the tracking system TRKSYS, a location or the tracking system TRKSYS, the relative spatial relationship between any two objects (e.g., tool to anatomy, tool to tracking system, HMD to localizer, etc.), surgical notifications or warnings, and the like. Surgical information may also include any surgical information described from the numerous examples above this paragraph, including information from the tracking device TD, etc.

At 306, a memory or database can optionally be utilized to save or retrieve information, configurations, or settings for the tracker device TD based on the various sources and information described above. The memory or database can be located at any suitable location such as a remote server, on the tracker device TD, or accessible by any one or more of the controllers TC, 26, 42, 54, 210. For instance, any surgical information can be utilized to query the database for trackable graphic TG settings and retrieve the settings from the database for presentation of the trackable graphic TG the digital display screen DS. The surgical information may any information described above, such as the identity of the surgical object being tracked, the identity of the tracking system TRKSYS tracking the tracker device TD, etc. Any trackable graphic TG settings can be retrieved such as the type or nature of trackable graphic TG, the respective location of the trackable graphic TG on the one or more display screens DS and the specification of a condition or timing to trigger presentation of the trackable graphic TG. At 308, given the numerous examples of information sources and information types, the operation or configuration of the tracker device TD and content displayed on the screen DS can be specified. Again, such configurations can be static, dynamically changing during surgery, automatically performed, or manually inputted.

### 4. Presentation of Other Content on Digital Display Screen of Tracker Device

The tracker device TD can additionally or alternatively present other content or information on the digital display screen DS. This content can be supplemental information or non-trackable information. This content can be presented with or without presenting the trackable graphic TG. The various controllers, sensors, configurations, information sources, information types, settings, conditions, and inputs described above can be used to detect, generate, configure, and present the following content on the display screen DS.

For example, human-readable information can be presented on the digital display screen DS. The human-readable information includes text, messages, or graphics that can be naturally read by the user and can be based on any information source or information type described above, such as but not limited to any one or more of: surgical information, operating instructions, information about the surgical object, an identity of the surgical object, a tracking status of the tracker device TD, an operation status of the tracking system TRKSYS, information about a surgical procedure or step of the surgical procedure, surgical plan information, and a warning or alert related to the surgical procedure.

Also, graphical information can be presented on the digital display screen DS. Graphical information includes pre-captured images, live-stream video feeds or real-time captured images, computer-generated 2D/3D graphics, and the like. The graphical information can be based on any information source or information type described above, such as but not limited to any one or more of: information of or about the surgical object, information of or about any surgical object, medical imaging data, video data from a camera, elements or icons of a graphical user interface, a video stream provided from a software application of a device in the operating room (e.g., the clinical application CA), a warning or alert information.

This content can be presented on the digital display screen DS according to numerous manners and forms. Content may be presented concurrently with the trackable graphic TG or at a different time from presentation of the trackable graphic TG. Content and the trackable graphic TG can be presented on one or multiple digital display screen DS concurrently. One display screen DS can present content and a second display screen DS can present the trackable graphics TG, at the same or separate times. Multiple digital display screens DS can present the same or distinct types of content. TG. The content can occupy an entirety or a portion of any digital display screen DS area. Content can span, extend across, or move across multiple digital display screens DS. When the digital display screen DS is curved, the content can be presented in a curved manner on the digital display screen DS and can move about the curved digital display screen DS. A configuration or geometry of the content may be configured to change, e.g., during use of the tracker device TD. One digital display screen DS may toggle on/off any content. Any one or more types of content may be swapped for presentation between one digital display screen DS and another digital display screen DS. The content may be static or may change pose on one or more digital display screens DS. The content may change size or shape on one or more digital display screens DS. The content may move about one or more digital display screens DS. The content may change form on one or more digital display screens DS. Any of these manners of controlling the content can be utilized individually or in combination. The user may utilize the graphical user interface GUI implemented by the tracker device TD to control settings to specify what, when, how, and where the content is/are presented on the digital display screen(s) DS.

i. Video Stream

The digital display screen DS can present a video stream or portion thereof that is directly obtained from a piece of equipment that hosts and runs a software application. Surgical information can be identified and/or extracted from any host system/device (e.g., in the operating room) that is configured to display the software application, such as the clinical application CA presented by the navigation system 20. The video stream may be wirelessly transmitted or communicated to the tracker device TD using a wired connection.

The host system/device 20 and respective software application can take various forms. For example, the host system/device 20 and software application can include any of: an endoscopic system that operates a software application for the endoscopic system; an imaging system (e.g., CT scanner) that operates a software application for the imaging system; a (CORE) console that operates a software application for operation of powered instruments; a surgical robot that operates a software application for controlling the surgical robot, a hand-held tool that operates a software application for controlling the hand-held tool, a surgical visualization system (e.g., arthroscope, ultrasound, laparoscope) that operates a software application for controlling the surgical visualization system, a surgical waste management system that operates a software application for controlling the surgical waste management system, a fluid management system that operates a software application for controlling the fluid management system, a sponge management system that operates a software application for controlling the sponge management system, a patient support apparatus that operates a software application for controlling the patient support apparatus, and the like.

In one implementation, any of the described controllers TC, 26, 42, 54, 210 can utilize a stream analyzer to process the video stream and recognize the surgical information therein by automatically identifying text presented by the software application. The stream analyzer can utilize any text recognition algorithm to perform this function, such as optical character recognition OCR, visual text recognition, scene text recognition, natural language processing NLP, any combination thereof, and the like. Additionally, or alternatively, any of the described controllers can utilize the stream analyzer to recognize the surgical information by automatically identifying imagery or graphics presented by the software application. The stream analyzer can utilize any image recognition algorithm to perform this function, such as segmentation, bounding boxes, pattern recognition, shape modeling, machine learning models, deep learning, neural networks, convolutional neural networks, any combination thereof, and the like. Additionally, or alternatively, the one or more controllers TC, 26, 42, 54, 210 can utilize the stream analyzer to recognize the surgical information by automatically identifying user inputs provided on the clinical application. Such inputs may include mouse movements or behavior, cursor selections, inputted text (e.g., using a keyboard), screen selections, icon selections, movement, or manipulation of graphical objects, such as scroll bars, up/down arrows, bone models, implants, and the like. Video streams can be analyzed according to any technique described in U.S. Provisional Patent App. No. 63/551,719, filed Feb. 9, 2024, and entitled “Extended Reality Systems and Methods for Surgical Applications”, the entire contents of which are hereby incorporated by reference.

The surgical information identified or extracted from the video stream by the one or more controllers TC, 26, 42, 54, 210 may include any information that may be relevant to the surgeon, patient, or surgical procedure. The surgical information may, but need not, be related to the process of actually performing surgery. The surgical information can be pre-operative surgical information. Alternatively, surgical information can include post-operative information, such as reports, etc. Examples of surgical information include but are not limited to: patient information, medical images (e.g., CT scan or volume, X-rays, etc.), surgical guidance information (e.g., tool interaction with target site), surgical planning information, an anatomical model, an implant model, a cut plan, a resection plan or volume, a virtual boundary VB or cutting boundary, surgical tool information, operating room or tool setup information, surgical step information, clinical application information, surgical alerts, notifications or warnings, error conditions, and the like. The surgical information can be a step of the surgical procedure. The step of the surgical procedure can include but are not limited to: a pre-operative planning step, an operating room setup step, an anatomical registration step, an intra-operative planning step, an anatomical preparation step, or a post-operative evaluation step. The surgical information detected can include initialization, progression, or completion of any surgical step. The surgical information detected can include a time component or duration defining presence or absence of any of the described surgical information.

The information identified and/or extracted from the software/clinical application of the host system/device depends on what the software/clinical application is configured to present. For example, the clinical application CA can have a plurality of different screens related to the surgical procedure, such as, but not limited to: “pre-op check,” “bone registration,” “intra-op planning,” “bone preparation” and “case completion.” Of course, the exact wording of the screen may vary depending on the clinical application CA use case. Each screen can have a screen identifier. Sometimes, the screen identifier can be a title of the screen, such as the “bone preparation” text. In one example, the one or more controllers TC, 26, 42, 54, 210 can utilize the stream analyzer to automatically identify the screen identifier of the active one of the screens of the clinical application CA. This can be performed by identifying the text of the title and/or by identifying any other graphic or text that can identify the contents of the screen. The information that identifies the screen can be located anywhere in the screen. Alternatively, the stream analyzer can specifically monitor changes in information provided only within a specified region on the screen. In another example, a detection region could be defined around a location on the screen where an icon or a visual indicator is/would be displayed. Additionally, or alternatively, the one or more controllers TC, 26, 42, 54, 210 can use the stream analyzer to identify or extract any information from the clinical application CA, regardless of identifying the specific screen on which such information is presented. For example, the stream analyzer can detect certain text/graphics that may be unique to the particular screen. The stream analyzer may detect the word “tibia” to understand the context or contents of the screen, e.g., that this screen involves bone preparation for the tibia (as compared to the femur, for example).

In some implementations, certain regions of the screen can be monitored from the video stream to detect surgical information that subsequently triggers monitoring or detection of another region of the screen. For example, the detection of specific text on the screen can trigger clipping or reproduction of a graphical part of the screen for presentation on the digital display screen DS of the tracker device TD. For example, the tracker device TD could reproduce a navigation guidance region on the digital display screen DS. The navigation guidance region can display one or more surgical objects tracked by a localizer 34 of a navigation system 20. Bounding boxes or detection regions can be used to define boundaries of the region to clip or reproduce for presentation on the digital display screen DS of the tracker device TD.

Advantageously, the described techniques can provide surgically relevant information, graphics, or video directly in front of the user/surgeon on the tracker device TD without needing to look away at an external display that is remotely located away from the surgical site. The tracker device TD will be located at the surgical site and its display screen DS will be readily visible to those at the surgical site. In turn, this technique can prevent impairing the user/surgeon's view and can minimize disruption or distraction to the user/surgeon.

Additionally, this video stream technique provides an intuitive and selective configuration dictating how information or how much information is displayed on the digital display screen DS of the tracker device TD. The described techniques can display the information that the user/surgeon needs when they need such information and where they need such information to be displayed. In turn, the described techniques avoid displaying an overwhelming amount of information and avoid displaying information in undesirable location. Also, by monitoring certain regions of the software application and/or clipping or reproducing certain portions of the software application, the described techniques advantageously improve performance of the system, speed of processing information, and recognition accuracy. Namely, by monitoring certain regions for information, the one or more controllers need not waste resources on monitoring the entirety of the video stream contents and can utilize its processing power for other purposes. Additionally, by clipping or reproducing only certain regions of the software application for presentation, the system can process and provide such information for display on the digital display screen DS of the tracker device TD much faster than if the entire software screen were to be reproduced. Hence, these techniques further reduce the latency in presentation of video data to the digital display screen DS of the tracker device TD.

### 5. Examples of Tracker Device in Surgical Settings

Examples of how the tracker device TD and tracker system TRKS may be used in the surgical setting will now be described with reference to FIGS. 7-11. These examples are provided for illustrative purposes and are not intended to limit the scope of the disclosure. Any information presented on the display screen(s) DS in these illustrations can alternatively be presented in any manner described above and are not limited to the examples shown.

FIG. 7 is a perspective view illustrating an example operation of the tracker device TD attached to the patient at the target site TS (e.g., femur bone). The digital display screen DS is presented in an enlarged view for simplicity in illustration. Here, the digital display screen DS simultaneously presents several types of information. One portion of the digital display screen DS is presenting the trackable graphic TG for tracking the patient anatomy to which the tracker device TD is attached. The trackable graphic TG is a QR code and may be deliberately sized to fit this region of the screen DS, yet large enough to facilitate accurate tracking. The remaining regions are reserved for displaying other content. The upper left region of the display screen DS presents surgical information in a human-readable manner. The patient's name, target anatomy and side, and type of procedure are shown. Such information can be presented for providing an extra level of confidence for the surgeon, e.g., to confirm that the surgical information and plan are correct. Any other type of human-readable surgical information can be presented in this manner. In the upper right region, the display screen DS presents a virtual 3D model of the patient anatomy subject to the procedure. This virtual model can be provided by the navigation system, e.g., from the surgical planning information or streamed from the clinical application CA. This model may be presented at certain times, e.g., only after the tracker device TD is registered to the anatomy.

FIG. 8 is a perspective view illustrating another example operation of the tracker device TD attached to a surgical tool 22. The surgical tool 22 is a hand-held saw device for manipulating the target site TS. More specifically, the tool 22 is for performing a resection of a tibia bone for a TKA surgery. Like the example of FIG. 7, the digital display screen DS simultaneously presents several types of information. The lower right portion of the digital display screen DS presents the trackable graphic TG for tracking the surgical tool 22 to which the tracker device TD is attached. The remaining regions are reserved for displaying other content. The lower left region of the digital display screen DS presents surgical information in a human-readable manner. The display DS presents the step of procedure, the tracking status of the tracker device TD, and the status of tool 22 operation. Such information can be presented for providing the surgeon with confirmation about the surgical step and tool operation. Again, any other type of human-readable surgical information can be presented in this manner.

In the upper half region, the display screen DS presents a portion of video stream that is directly obtained from the clinical application CA run by the navigation system 20, according to the techniques described above. For example, the tracker device TD may receive a real-time video stream of the clinical application CA that shows a guidance region presenting interaction between a virtual representation of the tool 22′ and a virtual representation of the target site TS' and a virtual boundary VB′ for guiding resection of the target site TS′. The relative spatial relationship between these virtual representations of the tool 22′ and target site TS' track, in real-time, the relative spatial relationship between the physical tool 22 and target site TS obtained from the localizer 34. The tracker device TD may display this guidance region in response to detection of certain events, e.g., in response to the one or more controllers: detecting the appropriate step of the procedure; detecting operation of the tool 22; and/or detecting text/graphics from the clinical application CA using the stream analyzer. Hence, the tracker device TD is configured to intelligently present information at appropriate or relevant times. Advantageously, as understood from this example, by having the tracker device TD attached to the surgical tool 22 (or any object controlled by the surgeon), the surgeon can easily visualize relevant information directly on the tracker device TD located directly next to the tool 22 without needing to look away, e.g., to an external monitor. This benefit of the tracker device TD helps reduce distractions during surgery and can help save time during surgery by dynamically providing any relevant information to the surgeon, without the surgeon asking for such information or looking for such information.

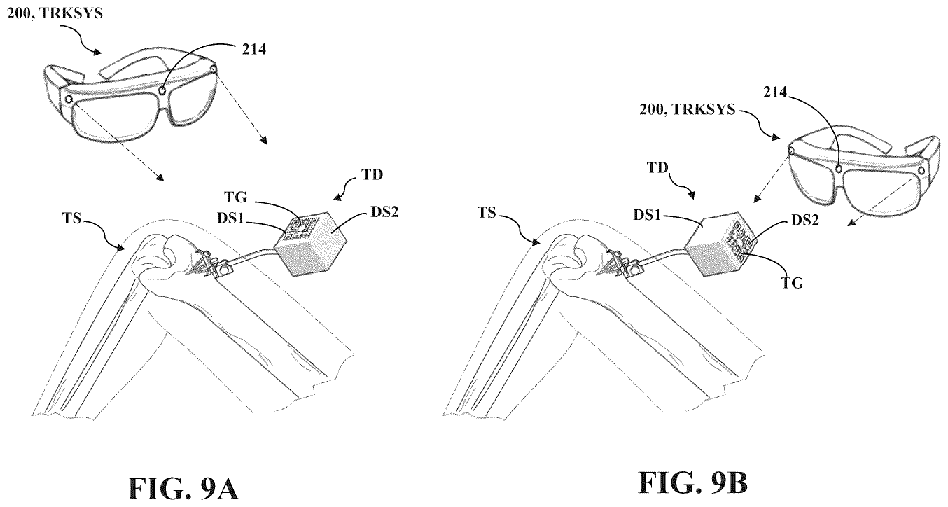

FIGS. 9A and 9B illustrate an example by which the head-mounted device HMD 200 (tracking system TRKSYS) observes one tracker device TD from different perspectives, according to one implementation. One tracker device TD is attached to the patient at the target site TS (e.g., femur bone) in both figures. The tracker device TD comprises at least two different display screens DS1, DS2. In this example, the display screens DS1, DS2 are provided on different sides of a cube-shaped tracker device TD. The display screen DS can be alternatively implemented as one or more curved screens (e.g., sphere or cylinder) to provide equivalent results. The HMD 200 may be used for extended reality purposes to visualize virtual graphics superimposed or combined with a real-world view. To facilitate such extended reality visualization, the HMD 200 uses its camera 214 to detect the trackable graphic TG presented on the display screen DS of the tracker device TD.

In FIG. 9A, the HMD 200 is viewing the target site TS from a first perspective and the HMD 200 detects the trackable graphic TG presented on the first display screen DS1. The tracker device TD intelligently presents the trackable graphic TG on the first display screen DS1 because the HMD 200 would be able to detect the trackable graphic TG on the first display screen DS1 from the first perspective. Similarly, the tracker device TD does not (yet) present the trackable graphic TG on the second display screen DS2 because the HMD 200 is not able to detect the trackable graphic TG on the second display screen DS2 from the first perspective.

In FIG. 9B, the HMD 200 is moved from the first perspective to a second perspective wherein the HMD 200 can no longer detect the trackable graphic TG presented on the first display screen DS1. As a result, the tracker device TD dynamically presents the trackable graphic TG on the second display screen DS2 to enable the HMD 200 to detect the trackable graphic TG. At the same time, the tracker device TD removes presentation of the trackable graphic TG on the first display screen DS1 knowing the HMD 200 would not be able to detect the trackable graphic TG on the first display screen DS1 from the second perspective.

Accordingly, the tracker device TD can make the intelligent determinations of when and where to display trackable graphics TG by detecting the presence and/or pose of the HMD 200 relative to the tracker device TD. Furthermore, the tracker device TD can conserve energy through such selective presentation of information. These techniques can be performed in numerous and ways using any of its sensors or components CAM, TFS, TX, PS, IMU, TC. The tracker device TD may detect a relative pose of the HMD 200 to the tracker device TD using one or more of its cameras CAM. For instance, the HMD 200 may be visible to the camera CAM associated with the first display screen DS1 and the tracker controller TC can detect the presence or pose of the HMD 200 and trigger presentation of the trackable graphic on the first display screen DS1. The tracker device TD can use the time-of-flight sensor TFS to detect the distance or relative spatial relationship between the HMD 200 and the tracker device TD. The proximity sensor PS can be used to detect presence or absence of the HMD 200. The transceiver TX can receive communication from the HMD 200 regarding its pose or active tracking status. Any of these techniques can be used individually or in combination to determine presence/absence of, or the spatial relationship of an object near the tracker device TD. Other techniques can be utilized as well.

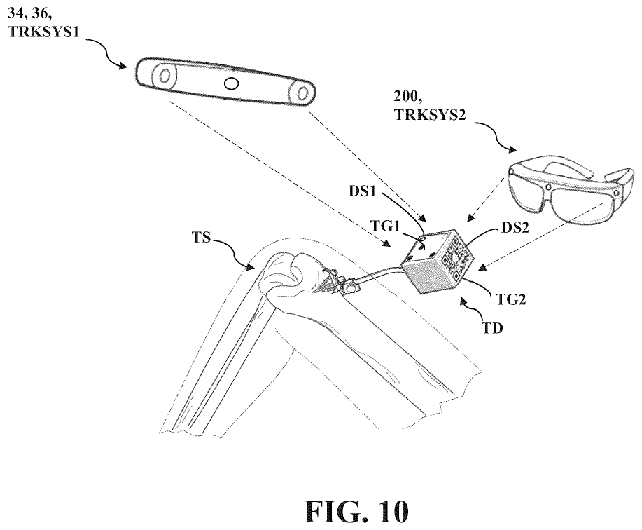

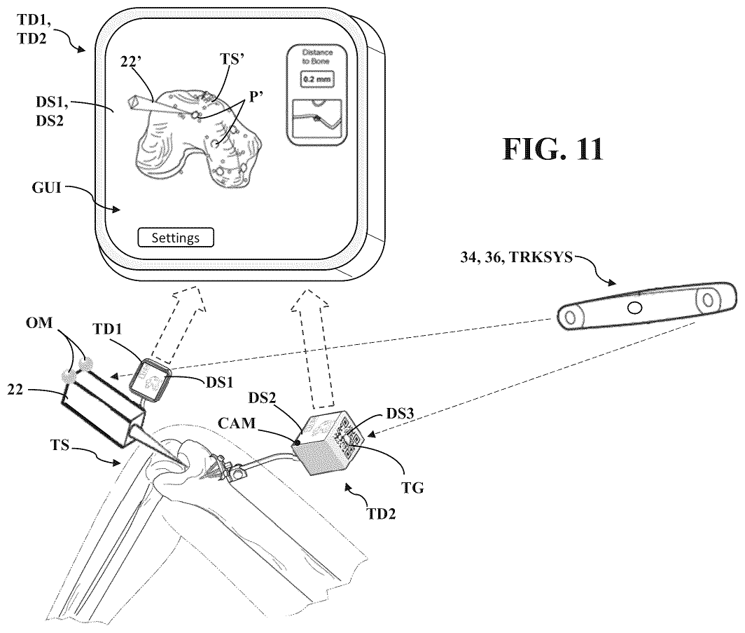

FIG. 10 is a perspective view illustrating an example of using the tracker device TD with multiple tracking systems TRKSYS. Specifically, in this example, one tracking system TRKSYS1 is the localizer 34/camera unit 36 of navigation tracking system 20 and the second tracking system TRKSYS2 is the HMD 200. The two tracking systems, TRKSYS1, TRKSYS2 observe one tracker device TD from different perspectives. The one tracker device TD is attached to the patient at the target site TS (e.g., femur bone) and once again comprises at least two different display screens DS1, DS2 provided on different sides of a cube-shaped tracker device TD. The display screen DS can be alternatively implemented as one or more curved screens (e.g., sphere or cylinder) to provide equivalent results.

Here, the two tracking systems, TRKSYS1, TRKSYS2 are viewing the tracker device TD from different perspectives. Like the example of FIG. 9, the tracker device TD intelligently determines when and where to display trackable graphics TG by detecting the presence/pose of the two tracking systems, TRKSYS1, TRKSYS2 relative to the tracker device TD. Moreover, in FIG. 10, the tracker device TD determines the type of each tracking system (i.e., navigation system localizer vs. HMD). The tracker device TD can make these determinations using any of the techniques described above. Accordingly, based on these determinations, the tracker device TD presents a first trackable graphic TG1 on the first display screen DS1 based on the presence and/or pose of the localizer 34 and presents a second trackable graphic TG2 on the second display screen DS2 based on the presence and/or pose of the HMD 200. Moreover, by knowing each type of tracking system, the trackable graphics TG1, TG2, can be customized for the localizer 34 and HMD 200, respectively. For example, the first trackable graphic TG1 can include digital fiducials that may better suited for detection by the optical sensors 40 of the localizer 34 while the second trackable graphic TG2 includes a QR code that may better be suited for detection by the camera 214 of the HMD 200. As the relative spatial relationship or presence between these three objects TD, TRKSYS1, TRKSYS2 changes, the tracker device TD can adapt by changing the location or type of trackable graphic TG on the display screens DS1, DS2 as described in FIG. 9. Advantageously, the one tracker device TD can be tracked by multiple tracking systems at the same or various times. For example, a curved or non-flat display of the tracker device TD can display multiple tracking graphics in different directions for the different tracking systems. These graphics can be in different parts of the display or overlapping for best viewing by each tracking system. In turn, the tracker device TD significantly reduces complexity and cost to the surgical system 10 by eliminating specialized trackers specifically configured for each tracking system.

Furthermore, in addition to facilitating tracking, the tracker device TD in this example may be utilized to initially register the tracking systems TRKSYS1, TRKSYS2 to each other, or to a common coordinate system. The tracker device TD may operate as a common registration device to facilitate registration of the tracking systems TRKSYS1, TRKSYS2. By each tracking system TRKSYS1, TRKSYS2 detecting the respective trackable graphic TG1, TG2, transforms can be computed from the HMD 200 to the localizer 34, and vice versa. Such transforms include the transform between each tracking system TRKSYS1, TRKSYS2 to the respective display screen DS1, DS2, and transforms based on the known relationship between each display screen DS1, DS2 and each trackable graphics TG1, TG2. The transforms can be combined to enable the HMD 200 and localizer 34 to be registered to one another. Accordingly, the tracker device TD also eliminates the need for specialized registration devices. Furthermore, the tracker device TD can be utilized for various purposes. In some cases, the tracker device TD can implement different operating modes, such as a tracking mode or registration mode, wherein the contents displayed on the display screen(s) DS are customized for the use of the tracking device TD (e.g., as a tracker or registration device).

FIG. 11 is a perspective view illustrating another example using multiple tracker devices TD1, TD2. The first tracker device TD1 with a first display screen DS1 is integrated with a surgical tool 22, e.g., a pointer or probe used for anatomical registration. The tool 22 also includes optical markers OM. The second tracker device TD2 is attached to the patient at the target site TS (e.g., femur bone) and once again comprises at least two different display screens DS2, DS3 provided on different sides of a cube-shaped tracker device TD. In this example, the tracking system TRKSYS is the localizer 34/camera unit 36 which can detect both infrared optical markers OM and visible light signals (e.g., from the digital display screens). Owing to the tracking system TRKSYS detecting the optical markers OM on the tool 22, the first tracker device TD1 displays surgical “non-trackable” content on the first display screen DS1. The second tracker device TD2 presents “non-trackable” content on the second display screen DS2 and presents a trackable graphic TG on the third display screen DS3. The tracking system detects the trackable graphic TG for detecting the second tracker device TD2 and for tracking the pose of the target site TS to which the second tracker device TD2 is attached. The first and second digital display screens DS1, DS2 of the respective first and second tracker devices TD1, TD2 are presenting the same content and are presented in an enlarged view for simplicity in illustration. Namely, both the first and second digital display screens DS1, DS2 present a portion of video stream that is directly obtained from the clinical application CA run by the navigation system 20, according to the techniques described above. For example, the tracker device TD may receive a real-time video stream of the clinical application CA that shows a guidance region presenting interaction between a virtual representation of the tool 22′ and a virtual representation (bone model) of the target site TS' and a virtual points P′ to be selected on the bone model for guiding anatomical registration. The relative spatial relationship between these virtual representations of the tool 22′ and target site TS' track, in real-time, the relative spatial relationship between the probe 22 and target site TS obtained from the localizer 34. The tracker device TD may display this guidance region in response to detection of certain events, e.g., in response to the one or more controllers: detecting the appropriate step of the procedure; detecting operation or active tracking of the tool 22; and/or detecting text/graphics from the clinical application CA using the stream analyzer.

Various scenarios may change how these tracker devices TD1, TD2, collectively present information. The tracker devices TD1, TD2 can communicate with each other and the one or more controllers can coordinate when, what, where, and how information is presented on the various display screens. For example, the surgical guidance region is simultaneously displayed on the tracker devices TD1, TD2. Knowing that this information is presented in a duplicative manner, the one or more controllers may take advantage of this duplicative content to present other material. For instance, if an HMD 200 were to be introduced during this process, the first tracker device TD1 could quickly change the content presented on the first display screen DS1 to be a trackable graphic TG customized for the HMD 200 instead of the surgical guidance region. This would enable the HMD 200 to track the tool 22, if it otherwise could not track the tool 22 using the optical markers OM. In other scenarios, if the localizer 34 temporarily loses sight of the optical markers OM on the tool 22, the first tracker device TD1 may react by immediately presenting a trackable graphic TG customized for the localizer 34 (e.g., such as an arrangement of digital fiducials mimicking the spatial geometry of the optical markers OM attached to the probe 22). Alternatively, the first tracker device TD1 may react by immediately presenting a trackable graphic TG and the second tracker device TD2 (having been registered to the target site TS) can use its camera CAM to detect the trackable graphic TG.

In other instances, the tracker devices TD1, TD2 could coordinate when, where and how to present the surgical content depending on the relative location and/or viewing perspective of the surgeon. The first tracker device TD1 may present the guidance region showing interactions between the virtual tool 22′ and the virtual target site TS′, while the second tracker device TD2 reserves the second display screen DS2 for displaying a sub-feature of this guidance region. For example, the second display screen DS2 could present the “distance to bone” sub-window shown in FIG. 11, or a warning or notification related to anatomical registration, e.g., “more points needed”, “registration complete”, etc. This coordination of multiple tracker devices TD can be utilized for any aspect of a surgical procedure beyond the examples shown and described. Similar techniques can be used for pre-operative planning, operating room setup screen, anatomical registration, intra-operative planning, anatomical preparation, post-operative evaluation, or the like.

While a tracking system TRKS is primarily used in the above examples, the disclosure is not limited to such. For example, any of the examples regarding content (visual or text) that is displayed by the tracker device TD can be presented or customized for a human observer. The tracker device TD is configured to use any of the described techniques to detect presence/absence of the human observer and/or a spatial relationship between the tracker device TD and the human observer. In response to such determinations, the tracker device TD is configured to modify what, where, when, and how content is presented on the digital display screen DS. For example, the tracker device TD may present content in response to detecting presence of the human observer, e.g., using the proximity sensor PS or camera CAM. The tracker device TD can change the location of the content on the display screen DS based on detecting changes in the relative spatial relationship between the tracker device TD and the human observer. The tracker device TD can move content from one display screen DS to another display screen DS based on detecting presence/absence or changes in the relative spatial relationship between the tracker device TD and the human observer. Other configurations are contemplated for modification of the displayed content to account for detectability of such content to human observers. Any of the techniques described above describing what, where, when, and how to modify the trackable graphic TG relative to tracking systems TRKSYS can similarly be utilized to specify what, where, when, and how to modify displayed content for human observers.

### 6. Example Technical Solutions and Advantages Provided by Tracker Device

The configurations and techniques involving the improved tracker device(s) TD, tracker system(s) TRKS, software or non-transitory computer readable medium(s), or methods that utilize and involve the digital display screen(s) DS provide significant advantages over conventional trackers and markers.

Firstly, the configurations and techniques described herein virtually eliminate the need to create or physically design a unique tracker for each different object that requires tracking. The tracker device TD can be compact, versatile, reusable, rechargeable, and does not require many parts to be assembled and separately sterilized. The tracker device TD can be used to track any surgical object in the same setting, merely by associating the tracker to the object or moving the tracker from one object to another. Also, one tracker device TD can be tracked by multiple tracking systems at the same or separate times. Accordingly, the improved tracker device TD significantly reduces complexity and cost to the surgical system.

Furthermore, the tracker device TD is intelligently controllable to dynamically adjust to various conditions. The tracker device TD exhibits adaptable and changing configurations to accommodate complex environmental conditions. The tracker device TD can display content or trackable graphics TG in a versatile manner, without being limited to a pre-set physical arrangement. The poses (position or orientation), shape, style, or arrangement of the content or trackable graphics TG can be actively controlled, changed, or moved on the digital display screen DS of the tracker device TD. One tracker device TD can coordinate what, when, how and where to present content on its display screen DS. Multiple tracker devices TD can coordinate what, when, how and where to present content on their respective display screens DS. The tracker device TD can adjust such content or trackable graphics TG to offset sub-optimal placement of the tracker device TD or maintain visibility of the information or trackable graphics TG relative to the tracking system TRKSYS. Content or trackable graphics TG can be visible in 360 degrees. As such, the improved tracker device TD is less susceptible to tracking inaccuracies and losing line-of-sight to the tracking system TRKSYS seeking to track the object.

Moreover, by utilizing the digital display screen(s) DS, the configurations and techniques described herein enable substantial new abilities to provide functionality beyond merely tracking the object. The tracker device TD facilitates user interaction with the device/system and enables communicating surgically meaningful content (such as text, graphics, or video) to the user and such content can be provided at surgically relevant times and customized to prevent display of excessive content. Multiple tracker devices TD can virtually replace a surgical navigation system by detecting one another and communicating and coordinating the display of content with each other. The described advantages are not intended to limit the scope of the invention. Other advantages can be readily understood from the detailed description and Figures.

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. A surgical system comprising:
a tracking system;
a robotic manipulator configured to support and move an end effector to perform a surgical procedure on a patient;
a tracker device being attached or attachable to the robotic manipulator, wherein the tracker device comprises a digital display screen; and
a controller in communication with the tracker device, wherein the controller is configured to instruct the digital display screen to present a computer-generated trackable graphic that is detectable by the tracking system to facilitate tracking of a pose of the robotic manipulator during the surgical procedure.

2. The surgical system of claim 1, wherein:
the robotic manipulator comprises a base, and a robotic arm coupled to the base; and
the tracker device is attached or attachable to one of: the base, the robotic arm, or the end effector.

3. The surgical system of claim 1, wherein the controller is configured to dynamically change a pose and/or configuration of the computer-generated trackable graphic on the digital display screen based on one or more of:
a relative spatial relationship between the tracker device and the tracking system; and/or
an absence or presence of line-of-sight between the tracker device and the tracking system.

4. The surgical system of claim 1, wherein the controller is configured to:
receive surgical information, wherein the surgical information comprises one or more of: information about the robotic manipulator, information about the surgical procedure or step of the surgical procedure, patient information, surgical plan information, surgeon preferences, a tracking status of the tracker device, an operation status of the tracking system, and a location or the tracking system; and
generate the computer-generated trackable graphic based on the surgical information.

5. The surgical system of claim 1, wherein the controller is configured to encode the computer-generated trackable graphic with time stamps to facilitate synchronization with the tracking system.

6. The surgical system of claim 1, wherein the computer-generated trackable graphic comprises a QR code or dynamic QR code.

7. The surgical system of claim 1, wherein the controller is configured to further instruct the digital display screen to present one or more of:
a tracking status of the tracker device;
an operation status of the tracking system; and/or
a status, step, notification or alert related to the surgical procedure.

8. The surgical system of claim 1, wherein the controller is configured to instruct the digital display screen to present a graphical user interface enabling a user to provide input to modify settings or operation of the tracker device or the robotic manipulator.

9. The surgical system of claim 1, wherein the controller is configured to further instruct the digital display screen to present graphical information, wherein the graphical information comprises one or more of;
information of, or related to, the robotic manipulator;
video data from a camera; and/or
a video stream provided from a software application of a device in an operating room.

10. The surgical system of claim 1, wherein the digital display screen is curved and the computer-generated trackable graphic is presented in a curved manner on the digital display screen.

11. The surgical system of claim 10, wherein;
the robotic manipulator comprises a base, and a robotic arm coupled to the base, wherein the robotic arm is formed of links; and
the digital display screen is wrapped partially or fully about one of the links of the robotic arm.

12. The surgical system of claim 1, wherein the digital display screen is a first digital display screen, and wherein the tracker device comprises a second digital display screen arranged to face a different direction from the first digital display screen.

13. The surgical system of claim 1, wherein the controller is further configured to instruct the digital display screen to present a video stream obtained by a camera source.

14. The surgical system of claim 1, wherein the tracker device further comprises an inertial sensor, and wherein the controller is configured to utilize measurements from the inertial sensor to perform one or more of the following:
change an orientation of the computer-generated trackable graphic on the digital display screen; and/or
detect an undesired motion of the tracker device.

15. The surgical system of claim 1, wherein the tracker device further comprises a camera configured to capture image or video data, and wherein the controller is configured to utilize the image or video data to perform one or more of the following:
detect an event and modify the computer-generated trackable graphic in response;
detect presence or absence of the robotic manipulator or the patient;
detect presence or absence of the tracking system;
present the image or video data on the digital display screen; and/or
detect a face of a user to authenticate use of the tracker device.

16. The surgical system of claim 1, wherein the tracker device further comprises an infrared or radio frequency transceiver, and wherein the controller is configured to utilize the transceiver to communicate to a transceiver of the tracking system.

17. The surgical system of claim 1, wherein the tracker device further comprises a proximity sensor, and wherein the controller is configured to utilize measurements from the proximity sensor to perform one or more of the following:
detect absence of environmental activity and in response place the tracker device or the digital display screen in a sleep mode to conserve energy; and/or
detect presence of environmental activity to ensure the tracker device or the digital display screen is active.

18. The surgical system of claim 1, wherein a drape is configured to cover the robotic manipulator and the digital display screen, wherein the drape comprises a transparent region to enable visibility of the digital display screen.

19. A surgical robotic system comprising:
a robotic manipulator comprising a robotic arm configured to support and move an end effector to perform a surgical procedure on a patient;
a tracker device being attached or attachable to the robotic manipulator, wherein the tracker device comprises a digital display screen; and
a controller in communication with the tracker device, wherein the controller is configured to instruct the digital display screen to present a computer-generated trackable graphic that is detectable by a tracking system to facilitate tracking of a pose of the robotic manipulator during the surgical procedure.

20. A tracker device for use with surgical system that includes a tracking system and a robotic manipulator, the tracker device comprising:
a body;
an attachment interface to facilitate coupling of the body to the robotic manipulator;
a digital display screen coupled to the body; and
a controller housed within the body and configured to instruct the digital display screen to present a computer-generated trackable graphic that is detectable by the tracking system to facilitate tracking of a pose of the robotic manipulator.
